(19) United States
(12) Patent Application Publication
(10) Pub. No.: US 2026/0285832 A1
(43) Pub. Date: Sep. 24, 2026

(54) SYNTHESIS OF MAVORIXAFOR AND INTERMEDIATES THEREOF

(71) Applicant: X4 Pharmaceuticals, Inc., Boston, MA (US)

(72) Inventors: Angelina Roberta Sekirnik, Logatec (SI); Art Taveras, Boston, MA (US); Feng Gao, Shanghai (CN); Zhigang Zhao, Shanghai (CN); Tao Xiao, Chengdu (CN); Shangfa Feng, Jiang Xi (CN)

(21) Appl. No.: 19/474,598

(22) PCT Filed: Apr. 12, 2024
(86) PCT No.: PCT/US2024/024331

§ 371 Date: Oct. 10, 2025

(30) Foreign Application Priority Data
Apr. 13, 2023 (WO) PCT/CN2023/088091

(51) Int. Cl. C07D 401/12

(52) U.S. Cl. CPC C07D 401/12

(57) ABSTRACT

The present invention relates to methods for synthesizing C-X-C receptor type 4 (CXCR4) inhibitor mavorixafor and to intermediates thereto.

### CROSS-REFERENCE TO RELATED APPLICATIONS

This application claims priority to International Patent Application Number PCT/CN2023/088091, filed Apr. 13, 2023, the contents of which are incorporated herein by reference.

### FIELD OF THE INVENTION

The present invention relates to methods for synthesizing mavorixafor, and to intermediates thereto.

### BACKGROUND OF THE INVENTION

C-X-C chemokine receptor type 4 (CXCR4), also known as fusin or cluster of differentiation 184 (CD 184), is a seven transmembrane G-protein coupled receptor (GPCR) belonging to Class I GPCR or rhodopsin-like GPCR family. Under normal physiological conditions. CXCR4 carries out multiple roles and is principally expressed in the hematopoietic and immune systems. CXCR4 was initially discovered as one of the co-receptors involved in human immunodeficiency virus (HIV) cell entry. Subsequent studies showed that it is expressed in many tissues, including brain, thymus, lymphatic tissues, spleen, stomach, and small intestine, and also specific cell types such as hematopoietic stem cells (HSC), mature lymphocytes, and fibroblasts. CXCL12, previously designated SDF-la, is the only known ligand for CXCR4. CXCR4 mediates migration of stem cells during embryonic development as well as in response to injury and inflammation. Multiple roles have been demonstrated for CXCR4 in human diseases such as cellular proliferative disorders, Alzheimer's disease, HIV, rheumatoid arthritis, pulmonary fibrosis, and others. For example, expression of CXCR4 and CXCL12 have been noted in several tumor types. CXCL12 is expressed by cancer-associated fibroblast (CAFs) and is often present at high levels in the tumor microenvironment (TME). In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver, and brain, which are sites of CXCL12 expression. CXCR4 is frequently expressed on melanoma cells, particularly the CD133+ population that is considered to represent melanoma stem cells; in vitro experiments and murine models have demonstrated that CXCL12 is chemotactic for such cells.

Peripheral leukocyte deficiency is a common feature of multiple diseases and may render affected individuals susceptible to infections, both common and opportunistic. The CXCR4 chemokine receptor regulates the trafficking of leukocytes among the bone marrow, blood, and lymphatic system (Al Ustwani 0, et al. Br J Haematol. 2014; 164:15-23). It is known to be involved with some immunodeficiencies such as WHIM syndrome, but not all human primary immunodeficiency diseases (PIDs) involve a mutation or alteration in CXCR4 function. PIDs comprise 330 distinct disorders with 320 different gene defects listed. Long considered as rare diseases, recent studies tend to show that they are more common than generally thought, if only by their rapidly increasing number. The International Union of Immunological Societies (IUIS) PID expert committee proposed a PID classification since 1999, which facilitates clinical research and comparative studies worldwide; it is updated every other year to include new disorders or disease-causing genes.

For example, neutropenia is a condition characterized by an abnormally low concentration of neutrophils circulating in the blood, and defined by an absolute neutrophil count (ANC) below 1500 cells/μL. Severe neutropenia (ANC <500 cells/μL) is a risk factor for susceptibility to bacterial infection. Neutrophils make up the majority of circulating white blood cells and play an important role in the body's defenses against bacterial or fungal pathogenic infections and in shaping the host response to infection. In addition, neutrophils participate in immune system homeostasis. Neutropenia can be divided into congenital (i.e., present at birth) and acquired. Additionally, neutropenia can be “acute” (transient, or temporary, often as a response to specific events that deplete the body of neutrophils, such as radiation or chemotherapy), or “chronic” (a long-term or long-lasting effect that may be due to the presence of genetic abnormalities).

Acute or transient neutropenia can be caused by infectious agents, such as the typhoid-causing bacterium Salmonella enterica; and cytomegalovirus, as well as chemical agents, including propylthiouracil; levamisole; penicillamine; clozapine; valproic acid; and cancer chemotherapy. Chronic neutropenia can be caused by genetic abnormalities (congenital neutropenia). Mutations in ELANE are the most common cause of congenital neutropenia. Other examples of genes that can be responsible for genetic causes of neutropenia include HAX1, G6PC3, WAS, SBDS, and others. In addition, some enzyme deficiencies can be associated with neutropenia such as glycogen storage disease 1b. Other causes of neutropenia include mitochondrial diseases, such as Pearson syndrome. Some autoimmune diseases, such as systemic lupus erythematosus (“SLE” or “lupus”) may be associated with neutropenia. Aplastic anemia, due to bone marrow failure, is associated with thrombocytopenia, anemia and neutropenia; Evans syndrome is characterized by autoimmune hemolytic anemia (AIHA) and immune thrombocytopenia (ITP) and/or immune neutropenia; and Felty's syndrome is characterized by rheumatoid arthritis, splenomegaly and neutropenia. Chronic neutropenia may also be the result of nutritional deficiencies, such as abnormally low levels of copper or Vitamin B12; or chronic infections, such as with human immunodeficiency virus (HIV), the agent that causes AIDS.

Neutropenia may be asymptomatic and often is only diagnosed fortuitously. Today, the standard treatment for severe neutropenia is administration of granulocyte colony-stimulating factor (G-CSF). Historically, neutropenia has been treated in a host of manners, including splenectomy, corticosteroids, androgens, and immunosuppressive and immune-modulating therapies. Currently, however, these treatments are generally not recommended except in cases where treatment with G-CSF is not effective. Dale et al. (2017) Curr. Opin. Hematol. 24:46-53; Sicre de Fontbrune et al. (2015) Blood 126:1643-1650. Other treatments for neutropenia can include bone marrow transportation and/or treatment with cord blood stem cells.

There exists a need for improved treatments capable of reversing peripheral leukocyte deficiencies, neutropenia, and other immune cell deficiencies. The present invention addresses this need and provides other related advantages. It has now been found that CXCR4 inhibitors such as mavorixafor (X4P-001) are useful for treating diseases and disorders related to immune-deficiencies, or diseases and disorders which present as imbalances in levels of one or more immune cells in a subject.

Furthermore, there is evidence implicating the CXCL12/CXCR4 axis in contributing to the loss or lack of tumor responsiveness to angiogenesis inhibitors (also referred to as “angiogenic escape”). In animal cancer models, interference with CXCR4 function has been demonstrated to alter the TME and sensitize the tumor to immune attack by multiple mechanisms such as elimination of tumor re-vascularization and increasing the ratio of CD8+ T cells to Treg cells. These effects result in significantly decreased tumor burden and increased overall survival in xenograft, syngeneic, and transgenic cancer models. See Vanharanta et al. (2013) Nat Med 19: 50-56; Gale and McColl (1999) BioEssays 21: 17-28; Highfill et al. (2014) Sci Transl Med 6: ra67; Facciabene et al. (2011) Nature 475: 226-230.

The above underscores the significant unmet need for CXCR4 inhibitors to treat the many diseases and conditions mediated by aberrant or undesired expression of the CXCR4 receptor, and to correct imbalances of cells of the immune system categorized as lymphocytes (T-cells, B-cells and NK cells), neutrophils, and monocytes/macrophages, for example in primary immunodeficiency disorders, neutropenia and cellular proliferative disorders.

### DETAILED DESCRIPTION OF THE INVENTION

Mavorixafor, and pharmaceutically acceptable compositions thereof, are effective as CXC receptor type 4 (CXCR4) inhibitors, and are useful for treating a variety of diseases, disorders, or conditions associated with CXCR4, such as hyperproliferative conditions including various cancers. In some embodiments, mavorixafor or a pharmaceutically acceptable salt thereof is prepared using a method disclosed herein. The methods and intermediates of the present invention are useful for preparing mavorixafor, which is described in, e.g., U.S. patent application Ser. No. 16/215,963 (U.S. Pat. No. 10,548,889), in the name of Brands, the entirety of which is incorporated herein by reference. The present invention relates to certain novel intermediates to the synthesis of mavorixafor that are easier to isolate, purify, and/or handle, and/or are more stable, and/or are more enantiomerically pure in comparison to corresponding intermediates in the existing methods. Furthermore, such intermediates enable methods of synthesis of mavorixafor that feature improved efficiency, reproducibility, purity, scalability, and manufacturability. In certain embodiments, the present compounds are generally prepared through intermediates A, B, and C, according to Scheme I set forth below:

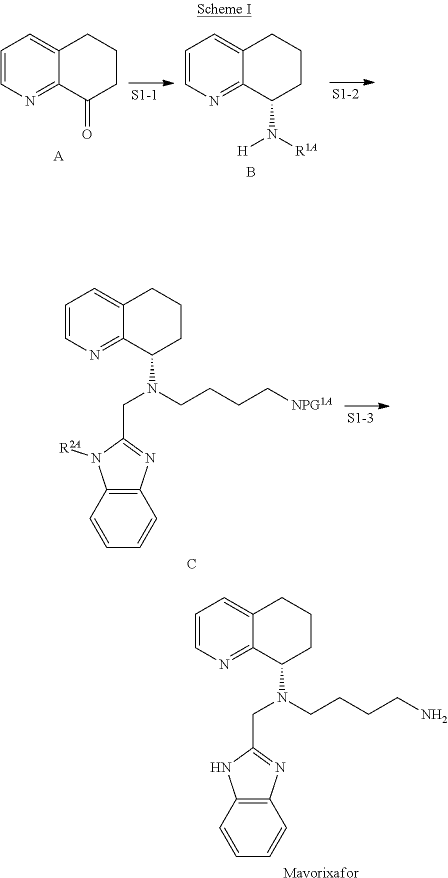

In Scheme I above, R^{1A} is H, a suitable amino protecting group, or chiral auxiliary; R^{2A} is H, or a suitable benzimidazole protecting group; and PG^{1A} is any combination of two amino protecting groups, a single amino protecting group and H, or two H. PG^{1A} may also be, taken with the nitrogen to which it is bound, a cyclic imide. Additionally. PG^{1A} may also be absent and a triple bond present between the corresponding nitrogen and the carbon to which the nitrogen is bound, such that the nitrogen and the carbon to which it is bound form a nitrile. In step S1-1, a ketone of the formula A is converted to a chiral compound of the formula B. In step S1-2, the compound of formula B is functionalized to prepare a compound of the formula C. In step S1-3, the compound of formula C is unmasked to prepare mavorixafor.

### Preparation of Mavorixafor

In one aspect, the present invention provides a method of preparing mavorixafor, or a pharmaceutically acceptable salt thereof, as set forth in Scheme II below.

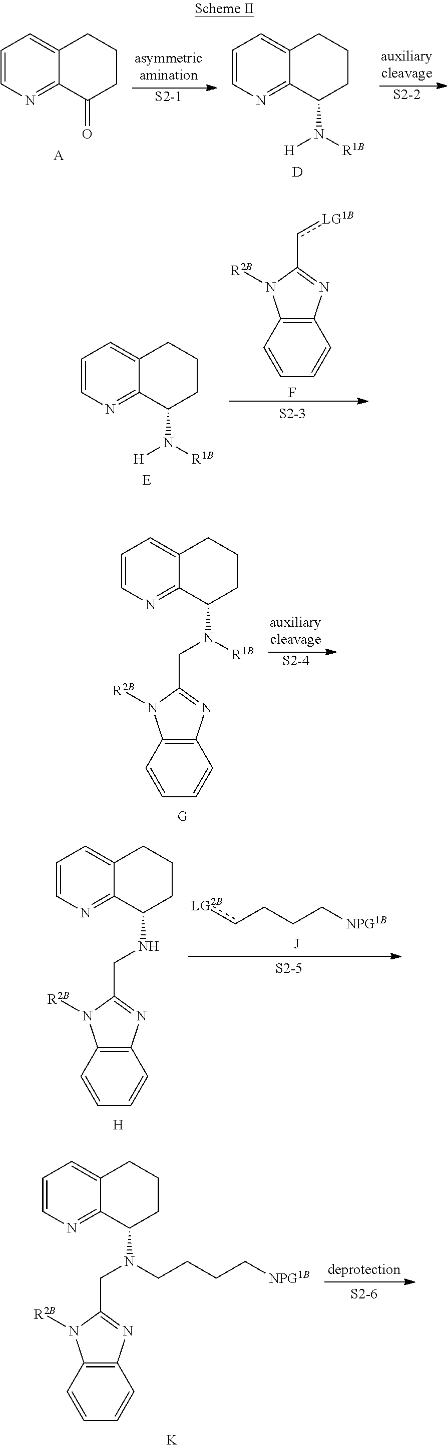

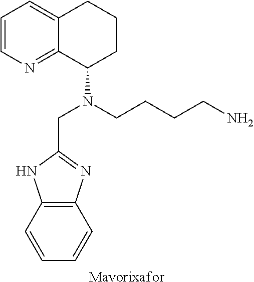

In one aspect, the present invention provides methods for preparing compounds of formulae D, E, F, G, H, J, K, and mavorixafor according to the steps depicted in Scheme II. In the compounds of the present formulae, R^{1B} is H, a suitable amino protecting group, or a chiral auxiliary; R^{2B} is H or a suitable benzimidazole protecting group; LG^{1B} and LG^{2B} independently are ═O, a suitable leaving group, or, taken with the carbon atoms to which they are bound, an acetal; and PG^{1B} is any combination of two amino protecting groups, any single amino protecting group and H, or two H. PG^{1B} may also be, taken with the nitrogen to which it is bound, a cyclic imide. Additionally, PG^{1B} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of a nitrile to an amino group.

Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference.

Cyclic imides are well known in the art and include those described in detail in Protecting Groups in Organic Synthesis, T. W. Greene and P. G. M. Wuts. 3^{rd} edition, John Wiley & Sons, 1999, the entirety of which is incorporated herein by reference. In some embodiments, PG^{1B} succinimide, maleimide, or phthalimide.

Acetals are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In some embodiments, LG^{1B}, taken with the carbon to which it is bound, is 1,3-dioxane or 1,3-dioxolane.

Suitable benzimidazole protecting groups are well known in the art and include those described in detail in Protecting Groups in Organic Synthesis, T. W. Greene and P. G. M. Wuts, 3^{rd} edition, John Wiley & Sons. 1999, the entirety of which is incorporated herein by reference. In certain embodiments. R^{2B}, taken with the nitrogen atom to which it is bound, is selected from, but are not limited to, aralkylamines, carbamates, allyl amines, amides, and the like, e.g., t-butyloxycarbonyl (Boc), trimethylsilylethoxymethyl (SEM), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, pivaloyl and the like.

Leaving groups are well known in the art and include those described in detail in Leaving group, Gold Book, IUPAC. 2009, ISBN 978-0-9678550-9-7, the entirety of which is incorporated herein by reference.

When >< is used to join two atoms, it denotes a double bond between the two atoms that may be of any geometry (cis or trans).

Chiral auxiliaries are well known in the art and include those described in detail in Key Chiral Auxiliary Applications, G. Roos, 2^{nd} edition, Academic Press, 2014; and Gnas et al, Synthesis, 12 (2006), 1899-1930, the entirety of which are incorporated herein by reference. In certain embodiments. R^{1B}, taken with the nitrogen atom to which it is bound, is selected from (S)-(−)-1-(4-methoxyphenyl)ethylamine, (S)-(−)-1-phenylethylamine, (R)-(+)-1-phenylethylamine and the like.

At step S2-1 an asymmetric reductive amination is carried out using the ketone group of compound A and to prepare a chiral compound of formula D. Methods of asymmetric reductive amination are well known to one of ordinary skill in the art; see Stereoselective Formation of Amines, Springer Berlin, 2014, ISBN 978-3-642-53928-2, the entirety of which is incorporated herein by reference. For additional examples of uses of chiral auxiliaries, see: Organic Process Research & Development, 2009, 13, 781-785; and European Journal of Medicinal Chemistry, 2018, 149, 30-44, the entirety of which are incorporated herein by reference. In certain embodiments, the solvent is selected from DCE, THF, MeCN, MeOH, or a combination thereof. In certain embodiments, the reducing agent is selected from NaCNBH₃ or NaBH₄. In certain embodiments, the solvent used is DCM. In certain embodiments, the reducing agent is NaBH(OAc)₃. In certain embodiments, the reaction is performed by allowing the amine and ketone to contact each other for 2 hours to 12 hours. In certain embodiments, the reaction is performed at 25° C. In certain embodiments, the reaction is carried out over 12 hours. In certain embodiments, the reaction is carried out over 16 hours. In certain embodiments, (S)-(−)-1-(4-methoxyphenyl)ethylamine is reacted with compound A to provide the compound D.

R^{1B} is cleaved at either step S2-2, S2-4, or S2-8. In some embodiments, at step S2-2, R^{1B} is cleaved to prepare a compound of formula E, such that R^{1B} is H. In some embodiments, R^{1B} is a chiral auxiliary. Methods of cleaving chiral auxiliaries are well known in the art and include those described in detail in Key Chiral Auxiliary Applications, G. Roos, 2^{nd} edition, Academic Press, 2014. In certain embodiments, TFA is used to cleave R^{1B}. In certain embodiments, DCM is used as a solvent. In certain embodiments, the cleavage of R^{1B} proceeds over 6 hours. In certain embodiments, the cleavage of R^{1B} proceeds at 25° C.

At step S2-3, the compound of formula E is alkylated with a compound of formula F to prepare a compound of formula G. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, the compound of formula E and the compound of formula F are coupled by Mitsunobu displacement, S_{N}2 reaction, or reductive amination. According to some embodiments, LG^{1B} is selected from mesylate, triflate, or tosylate. According to certain embodiments, LG^{1B} is ═O. According to some embodiments, LG^{1B} is, taken with the carbon atom to which it is bound, an acetal. According to some embodiments, LG^{1B} is hydroxyl. According to some embodiments, LG^{1B} is a halogen, such as chloro. According to certain embodiments, R^{2B} is SEM. According to other embodiments, R^{2B} is Boc. According to other embodiments, R^{2B} is CH₂OCH₂CH₃.

R^{1B} is cleaved at either step S2-2, S2-4, or S2-8. In some embodiments, R^{1B} is cleaved at step S2-4 to prepare a compound of formula H. R^{2B} may be cleaved concomitantly with R^{1B} in step S2-4, independently in step S2-4 or in step S2-6. According to some embodiments, R^{1B} is cleaved using TFA. According to some embodiments. DCM is a solvent.

At step S2-5, the amine of formula H is alkylated with a compound of formula J to prepare a compound of formula K. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, Mitsunobu displacement, reductive amination, or S_{N}2 reaction is used to couple the compound of formula H and the compound of formula J to produce a compound of formula K. According to some embodiments, LG^{2B} is selected from mesylate, triflate, or tosylate. According to some embodiments, LG^{2B} is ═O. According to some embodiments, LG^{2B} is, taken with the carbon atom to which it is bound, an acetal. According to some embodiments, LG^{2B} is hydroxyl. According to some embodiments, LG^{2B} is a halogen, such as bromo. In some embodiments, PG^{1B} is two Boc protecting groups, one Boc protecting group and H, or two H. In some embodiments, PG^{1B} is two Cbz protecting groups, one Cbz protecting group and H, or two H. In some embodiments, PG^{1B}, taken with the nitrogen to which it is bound, is a cyclic imide. In some embodiments, PG^{1B} is absent and a triple bond is present between the corresponding nitrogen and the carbon to which the nitrogen is bound, such that the nitrogen and the carbon to which it is bound form a nitrile. It is understood in the art the nitriles may function as masked amino groups.

At step S2-6, if R^{2B} is a benzimidazole protecting group and/or if PG^{1B}, taken with the nitrogen to which it is bound, is a cyclic imide, or if PG^{1B} includes any amino protecting group, or if PG^{1B} is absent and a triple bond is present between the corresponding nitrogen and the carbon to which the nitrogen is bound such that a nitrile is present, then the compound of formula K is deprotected to remove such protecting groups. Deprotection is understood to include reduction of the nitrile to an amino group. The protecting groups may be removed in a single step or in sequential steps. Deprotection methods of amine protecting groups and benzimidazole protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In some embodiments, hydrogenation is used. In some embodiments, TFA is used for the deprotection. In some embodiments, HCl in 1,4-dioxane is used for the deprotection. In some embodiments, HBr is used for the deprotections. In some embodiments, the deprotection proceeds over 8 hours. In some embodiments, the deprotection proceeds at 25° C. In some embodiments, DCM is a solvent.

In one aspect, the present invention provides an alternative method to prepare the compound of formula K according to Scheme II′ below. The compound of formula K may then be used to prepare mavorixafor according to Scheme II.

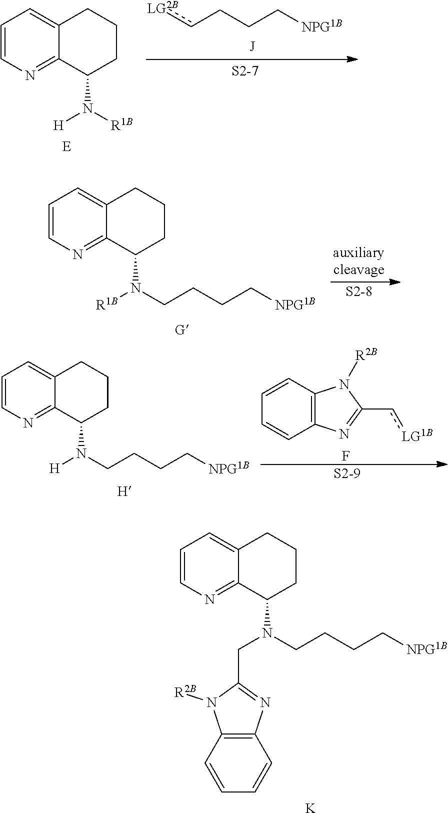

In one aspect, the present invention provides methods for preparing compounds of formulae G′, H′, and K according to the steps depicted in Scheme II′. In the compounds of the present formulae, R^{1B} is H, a suitable amino protecting group, or chiral auxiliary; R^{2B} is H or a suitable benzimidazole protecting group; LG^{1B} and LG^{2B} independently are ═O, a suitable leaving group, or, taken with the carbon atoms to which they are bound, an acetal; and PG^{1B} is any combination of two amino protecting groups, any single amino protecting group and H, or two H. PG^{1B} may also be, taken with the nitrogen to which it is bound, a cyclic imide. Additionally, PG^{1B} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S2-7, the compound of formula E is alkylated with the compound of formula J to prepare a compound of formula G′. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, Mitsunobu displacement, reductive amination, or S_{N}2 reaction is used to couple the compound of formula E and the compound of formula J to produce a compound of formula G′. According to some embodiments, LG^{2B} is selected from mesylate, triflate, or tosylate. According to some embodiments. LG^{2B} is ═O. According to some embodiments, LG^{2B} is, taken with the carbon atom to which it is bound, an acetal. According to some embodiments, LG^{2B} is hydroxyl. According to some embodiments, LG^{2B} is a halogen, such as bromo. In certain embodiments, PG^{1B} is two Boc protecting groups, one Boc protecting group and H. or two H. In certain embodiments, PG^{1B} is two Cbz protecting groups, one Cbz protecting group and H, or two H. In some embodiments, PG^{1B} is, taken with the nitrogen to which it is bound, a cyclic imide. In some embodiments, PG^{1B} is absent and a triple bond is present between the corresponding nitrogen and the carbon to which the nitrogen is bound, such that the nitrogen and the carbon to which it is bound form a nitrile.

R^{1B} is cleaved at either step S2-2, S2-4, or S2-8. In some embodiments, R^{1B} is cleaved at step S2-8 to prepare a compound of formula H′. According to some embodiments, the R^{1B} is cleaved using TFA. According to some embodiments, DCM is a solvent.

At step S2-9, the compound of formula H′ is alkylated with the compound of formula F to prepare a compound of formula K. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, the compound of formula H′ and the compound of formula F are coupled by Mitsunobu displacement, S_{N}2 reaction, or reductive amination. According to some embodiments, LG^{1B} is selected from mesylate, triflate, or tosylate. According to some embodiments, LG^{1B} is ═O. According to some embodiments, LG^{1B} is, taken with the carbon atom to which it is bound, an acetal. According to other embodiments, LG^{1B} is hydroxyl. According to some embodiments, LG^{1B} is a halogen, such as chloro. According to certain embodiments, R^{2B} is SEM. According to other embodiments, R^{2B} is Boc. According to other embodiments, R^{2B} is CH₂OCH₂CH₃.

In one aspect, the present invention provides a method of preparing mavorixafor, or a pharmaceutically acceptable salt thereof, as set forth in Scheme III below.

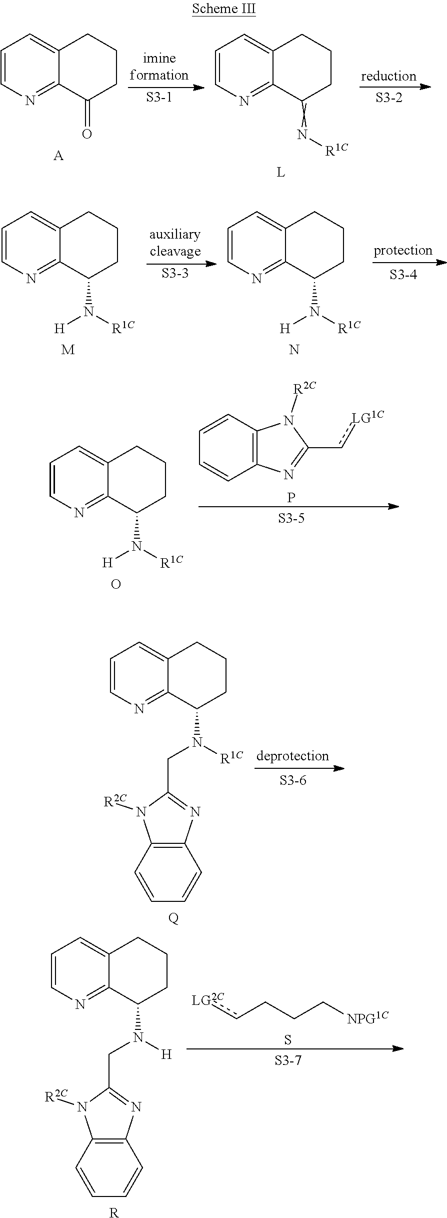

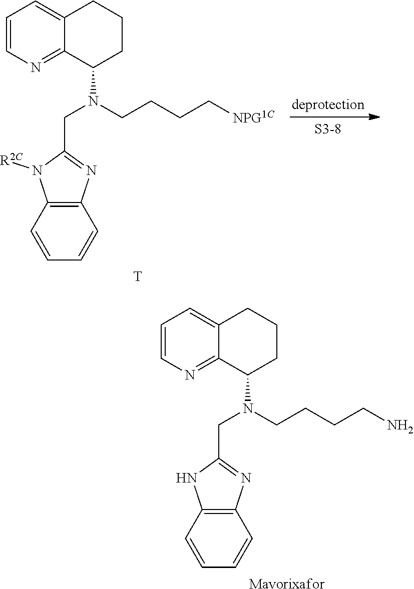

In one aspect, the present invention provides methods for preparing compounds of formulae L, M, N, O, P, Q, R, S, T and mavorixafor according to the steps depicted in Scheme II. In the compounds of the present formulae, R^{1C} is H, a chiral auxiliary, or a suitable amino protecting group; R^{2C} is H or a suitable benzimidazole protecting group; LG^{1C} and LG^{2C} independently are ═O, a suitable leaving group, or, taken with the carbon atoms to which they are bound, an acetal; and PG^{1C} is any combination of two amino protecting groups, any single amino protecting group and H, or two H. PG^{1C} may also be, taken with the nitrogen to which it is bound, a cyclic imide. Additionally, PG^{1C} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S3-1, a condensation reaction is carried out between the ketone in compound A and a chiral auxiliary to produce a compound of the formula L. Condensation reactions between ketones and chiral auxiliaries are well known in the art and include those described in detail in Synthesis and Applications of tert-Butanesulfonamide, 110 (2010), 3600-3740; or Application of Chiral Sulfinamides into Formation and Reduction of Sulfinylketimines to Obtain Valuable α-Chiral Primary Amines, European Journal of Organic Chemistry, 37 (2022), 5901-5916, the entirety of which are incorporated herein by reference. For additional examples of uses of chiral auxiliaries, see: Organic Process Research & Development, 2009, 13, 781-785; and European Journal of Medicinal Chemistry. 2018, 149, 30-44, the entirety of which are incorporated herein by reference. In some embodiments, the chiral auxiliary is (S)-(+)-p-toluenesulfinamide. In some embodiments, the chiral auxiliary is (S)-(−)-tert-butanesulfinamide. In some embodiments, the condensation is performed using Ti(OEt)₄. In some embodiments, the condensation is performed using Ti(i-PrO)₄. In some embodiments, THF or toluene is a solvent. In some embodiments, the reaction is performed at 60° C. In some embodiments, the reaction proceeds over the course of 12 hours.

In step S3-2, the imine moiety of the compound with formula L is reduced to prepare a compound with the formula M. Methods for reducing imines are well known to one of ordinary skill in the art. Methods to reduce of N-tert-butanesulfinyl imines are also well known in the art and include those described in detail in Synthesis and Applications of tert-Butanesulfonamide, 110 (2010), 3600-3740; or Application of Chiral Sulfinamides into Formation and Reduction of Sulfinylketimines to Obtain Valuable α-Chiral Primary Amines, European Journal of Organic Chemistry. 37 (2022), 5901-5916, the entirety of which are incorporated herein by reference. In certain embodiments, the reduction is performed using LiAlH₄. In certain embodiments, the reduction is performed using diisobutylaluminium hydride (DIBAL). In certain embodiments, the reduction is performed using NaBH₄. In certain embodiments, THF or MeOH is a solvent. In certain embodiments, the reaction is performed at 25° C. In certain embodiments, the reaction proceeds over 1 hour. In certain embodiments, diastereoselectivity can be controlled using zinc bromide as an additive.

R^{1C} is cleaved at either step S3-3, S3-6, or S3-10. In some embodiments, R^{1C} is cleaved at step S3-3 to prepare a compound of the formula N. In certain embodiments, R^{1C} is cleaved using HCl/dioxane. In certain embodiments, DCM is a solvent. In certain embodiments, the reaction proceeds over 1 hour. In certain embodiments, the reaction is performed at 25° C.

In step S3-4, the compound of formula N is optionally protected to prepare a compound of formula O. According to some embodiments, R^{1C} is 4-nitrobenzenesulfonyl. According to some embodiments, TEA is used. According to some embodiments, the reaction proceeds over 2 hours. According to some embodiments, 4-nitrobenzenesulfonyl chloride is used. According to some embodiments, the reaction proceeds at 25° C.

In step S3-5, the compound of formula O is alkylated with a compound of formula P to prepare a compound of Q. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, coupling of the compound of formula O with the compound of formula P to produce a compound of formula Q is carried out through Mitsunobu displacement, S_{N}2 reaction, or reductive amination. According to some embodiments, LG^{1C} is mesylate, triflate, or tosylate. According to some embodiments, LG^{1C} is ═O. According to some embodiments, LG^{1C} is, taken with the carbon atom to which it is bound, an acetal. According to another embodiment, LG^{1C} is hydroxyl. According to another embodiment, LG^{1C} is a halogen, such as chloro. According to some embodiments, the coupling between the compound of formula O and the compound of formula P is performed using Mitsunobu displacement. In some embodiments, the reaction is performed at 40° C. In some embodiments, the reaction begins at 0° C. and is then warmed to room temperature. In some embodiments, the reaction proceeds over 3 hours. In some embodiments, the reaction proceeds over 11.5 hours. According to some embodiments, R^{2C} is SEM.

In step S3-6, if R^{1C} is a chiral auxiliary or amino protecting group in the compound of formula Q, R^{1C} is cleaved to prepare a compound of formula R. R^{2C} may be cleaved concomitantly with R^{1C} in step S3-6, independently in step S3-6, or in step S3-8. In some embodiments, R^{1C} is cleaved with thiophenol. In some embodiments, R^{1C} is cleaved with 2-sulfanyl acetic acid.

In step S3-7, the amine of formula R is alkylated with a compound of formula S to produce a compound of formula T. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, coupling of the compound of formula R with the compound of formula S to produce the compound of formula T is carried out through Mitsunobu displacement, S_{N}2 reaction, or reductive amination. According to some embodiments, LG^{2C} is mesylate, triflate, ortosylate. According to some embodiments, LG^{2C} is ═O. According to some embodiments, LG^{2C} is, taken with the carbon atom to which it is bound, an acetal. According to some embodiments. LG^{2C} is hydroxyl. According to some embodiment, LG^{2C} is a halogen, such as bromo. In certain embodiments, PG^{1C} is two Boc protecting groups, one Boc protecting group and H, or two H. In certain embodiments, PG^{1C} is two Cbz protecting groups, one Cbz protecting group and H, or two H. In certain embodiments, PG^{1C} is, taken with the nitrogen to which it is bound, a cyclic imide. In some embodiments. PG^{1C} is absent and a triple bond is present between the corresponding nitrogen and the carbon to which the nitrogen is bound, such that the nitrogen and the carbon to which it is bound form a nitrile.

At step S3-8, if R^{2C} is a benzimidazole protecting group and/or if PG^{1C}, taken with the nitrogen to which it is bound, is a cyclic imide, or if PG^{1C} includes any amino protecting group, or if PG^{1C} is absent and a triple bond is present between the corresponding nitrogen and the carbon to which the nitrogen is bound such that a nitrile is present, then the compound of formula T is deprotected to remove such protecting groups. Deprotection is understood to include reduction of the nitrile to an amino group. The protecting groups may be removed in a single step or in sequential steps. Deprotection methods of amine protecting groups and benzimidazole protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In certain embodiments, hydrogenation is used for deprotection. In certain embodiments. HBr in AcOH is used. In certain embodiments, TFA is used.

In one aspect, the present invention provides an alternative method to prepare the compound of formula T according to Scheme III′ below. The compound of formula T may then be used to prepare mavorixafor according to Scheme III.

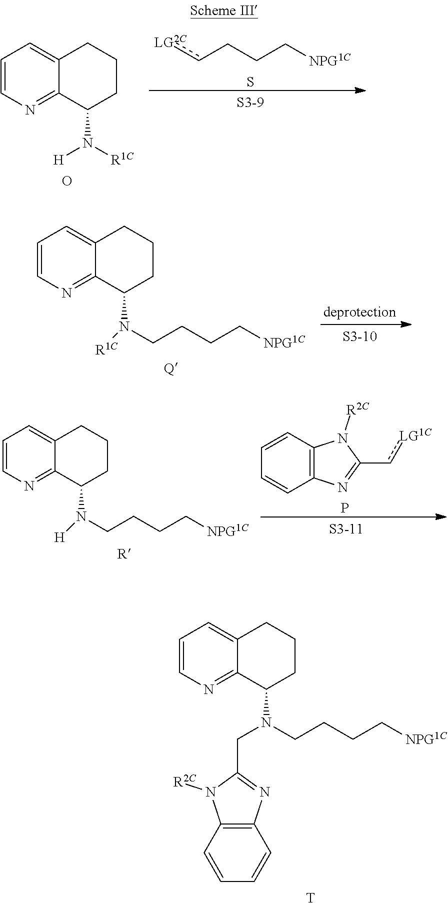

In one aspect, the present invention provides methods for preparing compounds of formulae Q′, R′, and T according to the steps depicted in Scheme III′. In the compounds of the present formulae, R^{1C} is H, a chiral auxiliary, or a suitable amino protecting group; R^{2C} is H or a suitable benzimidazole protecting group; LG^{1C} and LG^{2C} independently are ═O, a suitable leaving group, or, taken with the carbon atoms to which they are bound, an acetal; and PG^{1C} is any combination of two amino protecting groups, any single amino protecting group and H, or two H. PG^{1C} may also be, taken with the nitrogen to which it is bound, a cyclic imide. Additionally. PG^{1C} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S3-9, the compound of formula O is alkylated with a compound of formula S to produce a compound of formula Q′. Methods for N-alkylation of amines are well known to one of ordinary skill in the art. Exemplary reactions include those described in detail in Advanced Organic Chemistry: Reactions. Mechanisms, and Structure (3rd ed), Jerry March, New York: Wiley, ISBN 0-471-85472-7, the entirety of which is incorporated herein by reference. According to some embodiments, coupling of the compound of formula O with the compound of formula S to produce the compound of formula Q′ is carried out through Mitsunobu displacement, S_{N}2 reaction, or reductive amination. According to some embodiments, LG^{2C} is mesylate, triflate, or tosylate. According to some embodiments, LG^{2C} is ═O. According to some embodiments, LG^{2C} is hydroxyl. In some embodiments, LG^{2C} is, taken with the carbon atom to which it is bound, an acetal. According to another embodiment, LG^{2C} is hydroxyl. According to another embodiment, LG^{2C} is a halogen, such as bromo. In certain embodiments, PG^{1C} is two Boc protecting groups, one Boc protecting group and H, or two H. In certain embodiments, PG^{1C} is two Cbz protecting groups, one Cbz protecting group and H, or two H. In certain embodiments, PG^{1C} is, taken with the nitrogen to which it is bound, a cyclic imide. In some embodiments, PG^{1C} is absent and a triple bond is present between the corresponding nitrogen and the carbon to which the nitrogen is bound, such that the nitrogen and the carbon to which it is bound form a nitrile. It is understood in the art the nitriles may function as masked amino groups. According to some embodiments, PG^{1C} is H and Boc. According to some embodiments, the reaction is initiated at 0° C. and then warmed to room temperature. In some embodiments, the reaction proceeds over 2 hours.

In step S3-10, if R^{1C} in compound Q′ is an amino protecting group or a chiral auxiliary, R^{1C} is cleaved to produce a compound of formula R′. In some embodiments, thioglycolic acid and DBU are used.

In step S3-11, the compound of formula R′ is alkylated with the compound of formula P to prepare a compound of T. Methods for N-alkylation of amines are well known to one of ordinary skill in the art. Exemplary reactions include those described in detail in Advanced Organic Chemistry: Reactions, Mechanisms, and Structure (3rd ed.), Jerry March. New York: Wiley. ISBN 0-471-85472-7, the entirety of which is incorporated herein by reference. According to some embodiments, coupling of the compound of formula R′ with the compound of formula P to produce the compound of formula T is carried out through Mitsunobu displacement, S_{N}2 reaction, or reductive amination. According to some embodiments, LG^{1C} is mesylate, triflate, or tosylate. According to some embodiments, LG^{1C} is ═O. According to some embodiments, LG^{1C} is, taken with the carbon atom to which it is bound, an acetal. According to some embodiments, LG^{1C} is hydroxyl. According to some embodiments, LG^{1C} is a halogen, such as chloro. According to some embodiments, the coupling between the compound of formula R and the compound of formula P is performed using Mitsunobu displacement. According to some embodiments, R^{2C} is SEM. According to some embodiments, the coupling of the compound of formula R′ and the compound of formula P is performed by reductive amination.

In one aspect, the present invention provides a method of preparing mavorixafor, or a pharmaceutically acceptable salt thereof, as set forth in Scheme IV below.

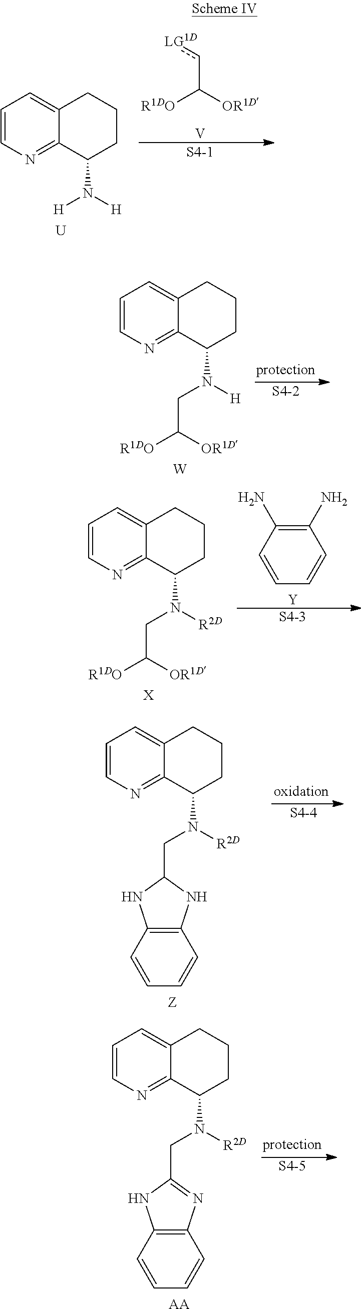

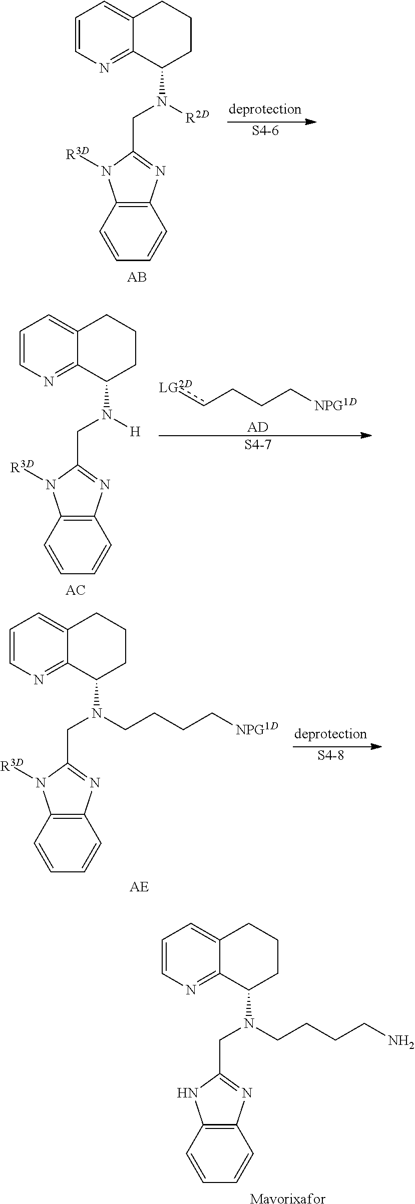

In one aspect, the present invention provides methods for preparing compounds of formulae V, W, X, Y, Z. AA, AB, AC. AD, AE, and mavorixafor according to the steps depicted in Scheme IV. In the compounds of the present formulae, R^{1D} and R^{1D′} are, taken together with the oxygen atoms to which they are bound and the carbon atom to which the oxygen atoms are bound, an acetal; R^{2D} is H, or a suitable amino protecting group; R^{3D} is H or a suitable benzimidazole protecting group; LG^{1D} and LG^{2D} independently are ═O, a suitable leaving group, or, taken with the carbon atoms to which they are bound, an acetal; and PG^{1D} is any combination of two amino protecting groups, any single amino protecting group and H. or two H. PG^{1D} may also be, taken with the nitrogen to which it is bound, a cyclic imide. Additionally. PG^{1D} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S4-1, a compound of formula U is coupled with a compound of formula V to prepare a compound of formula W. In some embodiments, R^{1D} and R^{1D′} are, taken together with the oxygen atoms to which they are bound and the carbon atom to which the oxygen atoms are bound, an acetal. In some embodiments, R^{1D} and R^{1D′}, taken with the oxygen atoms to which they are bound and the carbon to which the oxygens are bound, are 1,3-dioxolane or 1,3-dioxane. In some embodiments, R^{1D} and R^{1D′} are each ethyl. In some embodiments, LG^{1D} is a halogen, such as bromo. In some embodiments, LG^{1D} is hydroxyl. In some embodiments, LG^{D} is ═O.

In step S4-2, the amino of the formula W is protected to prepare a compound of the formula X. In some embodiments. R^{2D} is Cbz.

In step S4-3, the compound of the formula X is reacted with a compound of formula Y to prepare a compound of formula Z. In some embodiments, TsOH is used.

In step S4-4, the compound of formula Z is oxidized to prepare a compound of formula AA. In some embodiments 12 is used.

In step S4-5, the compound of formula AA is protected to prepare a compound of formula AB. In some embodiments, R^{3D} is SEM. According to some embodiments, R^{3D} is PMB. According to some embodiments, R^{3D} is Cbz. According to some embodiments, R^{3D} is Boc.

In step S4-6, if R^{2D} is an amino protecting group, then R^{2D} is removed from the compound of formula AB to prepare a compound of the formula AC. In some embodiments, hydrogenation using H₂ and Pd/C is employed.

In step S4-7, the compound of formula AC is alkylated with a compound of formula AD to produce a compound of formula AE. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. According to some embodiments, coupling of the compound of formula AC with the compound of formula AD to produce the compound of formula AE is carried out through Mitsunobu displacement, S_{N}2 reaction, or reductive amination. According to some embodiments, LG^{2D} is mesylate, triflate, or tosylate. According to some embodiments, LG^{2D} is ═O. According to some embodiments, LG^{2D} is, taken with the carbon atom to which it is bound, an acetal. According to some embodiments, LG^{2D} is hydroxyl. According to some embodiments, LG^{2D} is a halogen, such as bromo. In certain embodiments, PG^{1D} is two Boc protecting groups, one Boc protecting group and H, or two H. In certain embodiments, PG^{1D} is two Cbz protecting groups, one Cbz protecting group and H, or two H. In certain embodiments, PG^{1D} is, taken with the nitrogen to which it is bound, is a cyclic imide. In some embodiments, PG^{1D} is absent and a triple bond is present between the corresponding nitrogen and the carbon to which the nitrogen is bound, such that the nitrogen and the carbon to which it is bound form a nitrile.

In step S4-8, if R^{3D} is a benzimidazole protecting group and/or if PG^{1D}, taken with the nitrogen to which it is bound, is a cyclic imide, or if PG^{1D} includes any amino protecting group, or if PG^{1D} is absent and a triple bond is present between the corresponding nitrogen and the carbon to which the nitrogen is bound such that a nitrile is present, then the compound of formula AE is deprotected to remove such protecting groups. Deprotection is understood to include reduction of the nitrile to an amino group. The protecting groups may be removed in a single step or in sequential steps. Deprotection methods of amine protecting groups and benzimidazole protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference.

In one aspect, the present invention provides an alternative method to prepare the compound of formula AE according to Scheme IV′ below. The compound of formula AE may then be used to prepare mavorixafor according to Scheme IV.

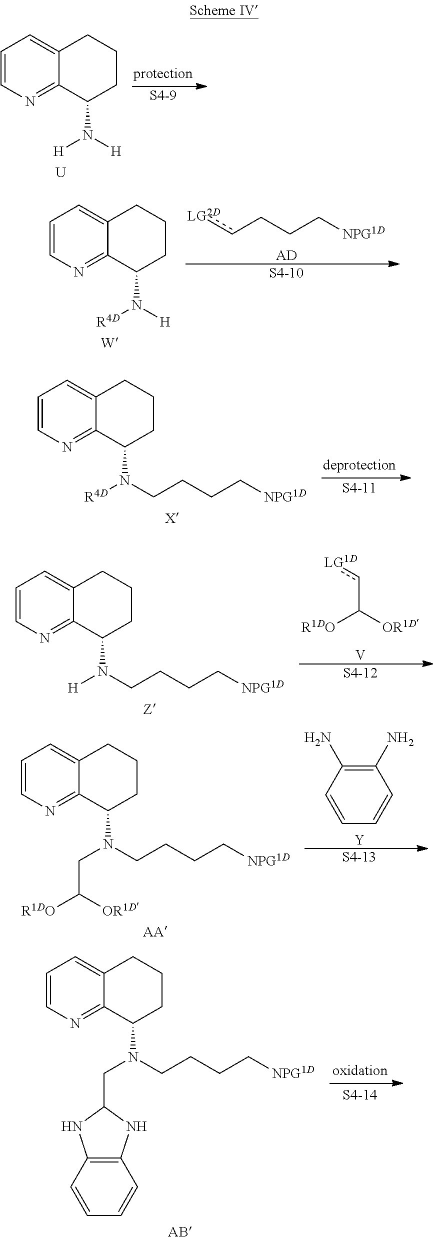

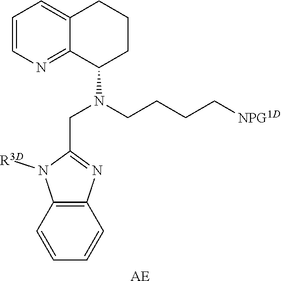

In one aspect, the present invention provides methods for preparing compounds of formulae W′, X′, Z′, AA′, and AB′ according to the steps depicted in Scheme IV′. In the compounds of the present formulae, R^{1D} and R^{1D′} are, taken together with the oxygen atoms to which they are bound and the carbon atom to which the oxygen atoms are bound, an acetal; R^{4D} is H, or a suitable amino protecting group; R^{3D} is H or a suitable benzimidazole protecting group; LG^{1D} and LG^{2D} independently are ═O, a suitable leaving group, or, taken with the carbon atoms to which they bound, an acetal; and PG^{1D} is any combination of two amino protecting groups, any single amino protecting group and H, or two H. PG^{1D} may also be, taken with the nitrogen to which it is bound, a cyclic imide. Additionally, PG^{1D} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S4-9, the compound of the formula U is protected to prepare a compound of the formula W′. In some embodiments, R^{4D} is Cbz.

In step S4-10, the compound of the formula W′ is alkylated with the compound of formula AD to prepare a compound of the formula X′. Methods for N-alkylation of amines are well known to one of ordinary skill in the art. Exemplary reactions include those described in detail in Advanced Organic Chemistry: Reactions, Mechanisms, and Structure (3rd ed.), Jerry March. New York: Wiley. ISBN 0-471-85472-7, the entirety of which is incorporated herein by reference. According to some embodiments, coupling of the compound of formula W′ with the compound of formula AD to produce the compound of formula X′ is carried out through Mitsunobu displacement, S_{N}2 reaction, or reductive amination. According to some embodiments, LG^{2D} is mesylate, triflate, or tosylate. According to some embodiments, LG^{2D} is ═O. According to some embodiments, LG^{2D} is, taken with the carbon atom to which it is bound, an acetal. According to some embodiments, LG^{2D} is hydroxyl. According to some embodiments, LG^{2D} is a halogen, such as bromo. In some embodiments, PG^{1D} is two Boc protecting groups, one Boc protecting group and H. or two H. In some embodiments, PG^{1D} is two Cbz protecting groups, one Cbz protecting group and H, or two H. In some embodiments, PG^{1D} is a cyclic imide. In some embodiments, PG^{1D} is absent and a triple bond is present between the corresponding nitrogen and the carbon to which the nitrogen is bound, such that the nitrogen and the carbon to which it is bound form a nitrile.

In step S4-11, if R^{4D} is an amino protecting group, the R^{4D} is cleaved from the compound of formula X′ to prepare a compound of the formula Z′. In some embodiments, R^{4D} is cleaved using H₂ and Pd/C.

In step S4-12, the compound of formula Z′ is coupled with the compound of formula V to prepare a compound of the formula AA′. In some embodiments, R^{1D} and R^{1D′} are, taken together with the oxygen atoms to which they are bound and the carbon atom to which the oxygen atoms are bound, an acetal. In some embodiments, R^{1D} and R^{1D′}, taken with the oxygen atoms to which they are bound and the carbon to which the oxygens are bound, are 1,3-dioxolane or 1,3-dioxane. In some embodiments, R^{1D} and R^{1D′} are each ethyl. In some embodiments, LG^{1D} is a halogen, such as bromo. In some embodiments, LG^{1D} is hydroxyl. In some embodiments, LG^{1D} is ═O.

In step S4-13, the compound of formula AA′ is coupled with the compound of formula Y to prepare a compound of formula AB′. In some embodiments, TsOH is used for the coupling.

In step S4-14, the compound of formula AB′ is oxidized to prepare the compound of formula AE. In some embodiments, 12 is used for the oxidation.

### EXEMPLIFICATION

### Example 1: First Synthesis of Mavorixafor

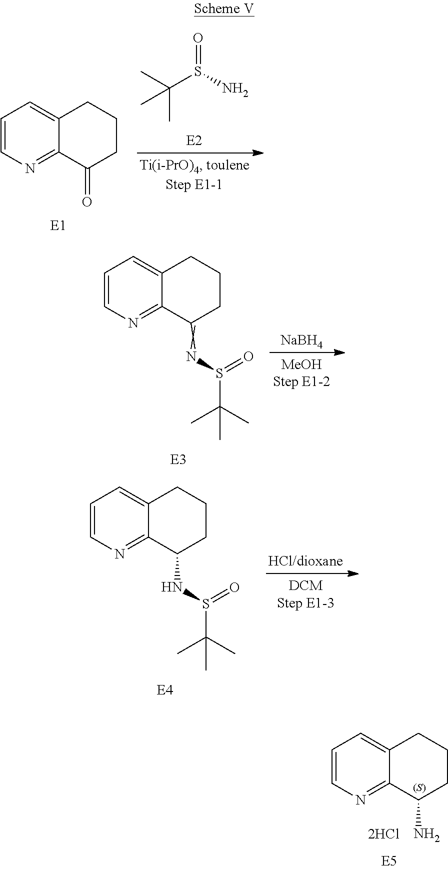

### Step E1-1: Preparation of Imine E3

To solution of compound E1 (50.0 g, 339 mmol, 1.00 eq.) in THF (500 mL) was added Ti(i-PrO)₄ (386 g. 1.36 mol, 401 mL, 4.00 eq.) and compound E2 (82.3 g, 679 mmol, 2.00 eq.) and was stirred for 12 h at 60° C. TLC (plate 1. Petroleum ether:Ethyl acetate=1:1, R_{f}(R1)=0.13, R_{f}(P1)=0.58) indicated compound E1 was consumed completely and anew spot was detected. The reaction solution was quenched by NaHCO₃ (500 mL) at 0° C. and extracted With ethyl acetate 600 mL (200 mL*3). The combined organic layers were washed with brine 150 mL (50.0 mL*3), filtered and concentrated under reduced pressure to give the crude compound. The residue was purified by column chromatography (SiO₂, petroleum ether:ethyl acetate=1:0 to 0:1). Compound E3 (22.4 g) was obtained as a brown oil and used for the next step.

### Step E1-2: Reduction of E3.

To a solution of compound E3 (22.4 g, 89.47 mmol, 1.00 eq.) in THF (200 mL) was added NaBH₄ (9.69 g, 256 mmol, 2.86 eq.) at 0° C. and then stirred at 25° C. for 1 h. TLC (plate 1, petroleum ether:ethyl acetate=1:1, R_{f}(R1)=0.58, R_{f}(P1)=0.05) indicated compound E3 was consumed completely and a new spot was detected. The reaction solution was quenched with H₂O (200 mL) and extracted with ethyl acetate 600 mL (200 mL*3). The organic layers were washed with H₂O 300 mL (100 mL*3), dried over Na₂SO₄, and concentrated under reduced pressure to afford compound E4 (15.2 g. crude) as a yellow oil, which was used for the next step directly.

### Step E1-3: Formation of Amine E5

To solution of compound E4 (15.2 g, 60.2 mmol, 1.00 eq.) in DCM (150 mL) was added HCl/dioxane (4.00 M, 150 mL, 10.0 eq.) and then stirred for 1 h at 25° C. LC-MS indicated compound E4 was consumed completely and a main peak with the desired mass was detected. The reaction solution was concentrated to remove HCl/dioxane to give the crude product. The crude product was dispersed in DCM (100 mL), followed by addition of TEA (13.0 mL) and Boc₂O (13.0 mL) and stirred for 1 h. The mixture was quenched with H₂O (100 mL) and extracted with DCM (100 mL*2). The combined organic was concentrated under reduced pressure and purified by column chromatography (SiO₂, petroleum ether:ethyl acetate=1:0 to 3:1, plate 1, petroleum ether:ethyl acetate=1:1, R_{f}(P1)=0.58). Then the residue was dissolved in DCM (100 mL) and HCl/dioxane (100 mL) was added and the mixture was stirred for 1 h. After 1 h, the solution was concentrated under reduced pressure to yield crude compound E5 (10.0 g, crude, 2HCl) as white solid, confirmed by LC-MS.

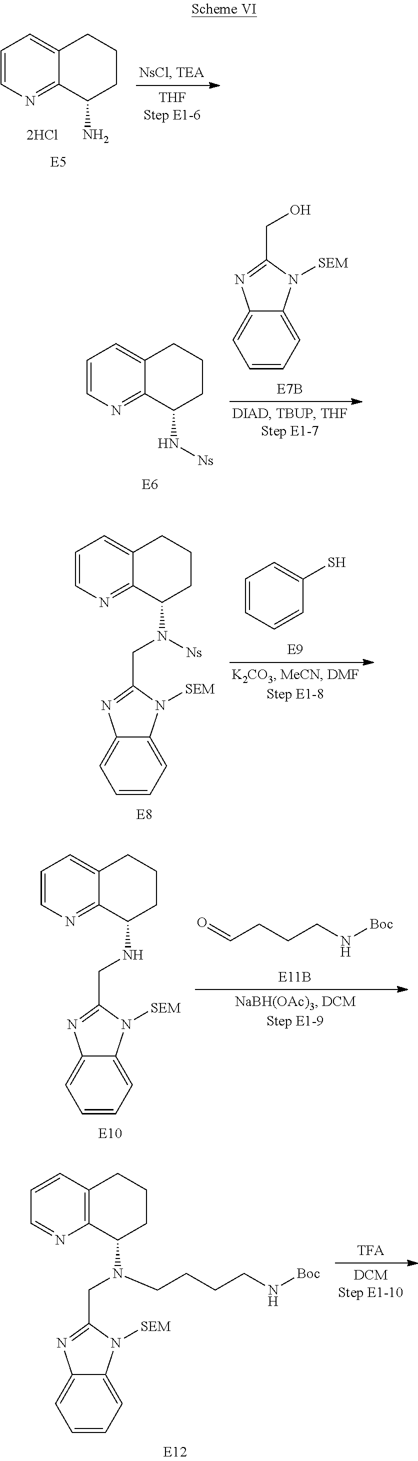

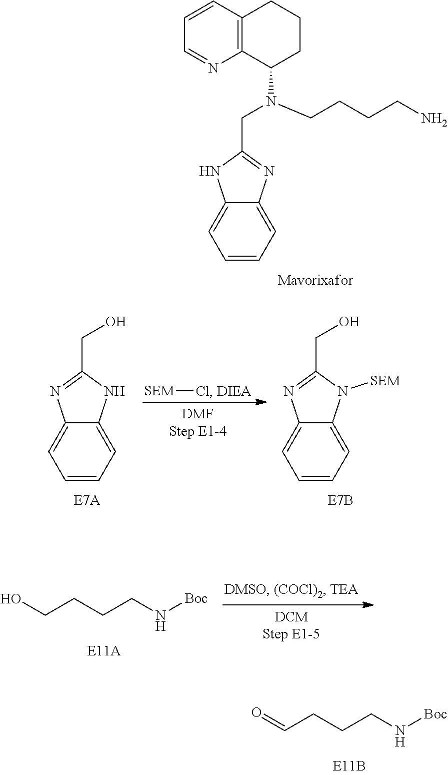

### Step E1-4: Protection of Benzimidazole E7A

To a solution of E7A (25.0 g, 168 mmol, 1.00 eq.) and DIEA (52.3 g, 404, mmol, 70.5 mL, 2.40 eq.) in DMF (400 mL) was added SEM-Cl (32.3 g, 194 mmol, 34.3 mL. 1.15 eq.) at 25° C. and the mixture was stirred at 60° C. for 2 h. LC-MS indicated E7A was consumed completely and a main peak with the desired mass was detected. The reaction solution was quenched by H₂O (200 mL) and extracted with ethyl acetate (150 mL*2). The combined organic layers were washed with brine (75.0 mL*2), dried over Na₂SO₄, filtered, and concentrated to give the crude product under reduced pressure. The residue was purified by column chromatography (SiO₂, petroleum ether:ethyl acetate=1:0 to 0:1, plate 1, petroleum ether:Ethyl acetate=1:1, R_{f}(P1)=0.60). E7B (28.0 g, 100 mmol, 59.6% yield) was obtained as a brown oil. ¹H NMR (d₆-DMSO, 400 MHz): δ 7.65-7.56 (m, 2H), 7.31-7.17 (m, 2H), 5.69 (s, 2H), 4.75 (s, 1H), 4.73 (s, 1H), 3.57-3.47 (m, 2H), 0.86-0.79 (m, 2H), −0.10 (s, 9H).

### Step E1-5: Oxidation of Alcohol E11A

To a solution of (COCl)₂ (6.71 g, 52.8 mmol, 4.63 mL, 1.00 eq.) in DCM (80.0 mL) was added DMSO (4.95 g. 63.4 mmol, 4.95 mL, 1.20 eq.) in DCM (60.0 mL) dropwise at −78° C. and stirred for 10 min under N2. Then. E11A (10.0 g. 52.8 mmol. 1.00 eq.) in DCM (60.0 mL) was added dropwise at −78° C. and stirred for 20 min. Finally, TEA (16.0 g, 158 mmol, 22.0 mL, 3.00 eq.) was added dropwise at −78° C. and the mixture was warmed up to 20° C. and stirred for 30 min under N2. TLC (Petroleum ether:Ethyl acetate=1:1. R_{f}(R1)=0.30, R_{f}(P1)=0.60, I₂) indicated E11A was consumed completely and a new spot was detected. The reaction solution was quenched with H₂O (10.0 mL), extracted with DCM (10.0 mL), and the organic layers were concentrated to give the crude product without purification. Compound E11B (9.00 g, crude) was obtained as yellow oil and used for the next step directly.

### Step E1-6: E5 Amine Protection

To a solution of compound E5 (10.0 g, 45.2 mmol, 1.00 eq., 2HCl) and TEA (6.86 g, 67.8 mmol, 9.44 mL, 1.50 eq.) in THF (60.0 mL) was added 4-nitrobenzenesulfonyl chloride (10.5 g, 47.4 mmol, 1.05 eq.), and then stirred at 25° C. for 2 h. TLC (plate 1, petroleum ether:ethyl acetate=1:1, R_{f}(R1)=0.00, R_{f}(P1)=0.37) indicated compound E5 was consumed completely and a new spot was detected. The reaction solution was quenched with H₂O (100 mL) and extracted with ethyl acetate 150 mL (50.0 mL*3). The combined organic layers were washed with H₂O (50.0 mL), dried over Na₂SO₄, filtered, and concentrated under reduced pressure to give the crude product. The residue was purified by column chromatography (SiO₂, petroleum ether:ethyl acetate=1:0 to 0:1). Compound E6 (12.0 g, 36.0 mmol, 79.6% yield) was obtained as white-yellow solid. ¹H NMR (d₆-DMSO. 400 MHz): δ 8.41-8.09 (m, 5H), 7.47 (d, J=7.2 Hz, 1H), 7.13 (dd, J₁=7.6 Hz, J₂ 4.6 Hz, 1H), 4.52-4.42 (m, 1H), 2.79-2.60 (m, 2H), 2.00-1.62 (m, 4H). LC-MS: (M+H)+=334.0.

Step E1-7: E6 Condensation with Aldehyde E7B

A solution of DIAD (10.9 g, 54.0 mmol, 10.4 mL, 1.50 eq.) and tributylphosphane (14.5 g. 71.9 mmol, 17.7 mL. 2.00 eq.) in THF (150 mL) was stirred at 25° C. for 0.5 h, followed by addition of E6 (12.0 g, 36.0 mmol, 1.00 eq.) and E7B (15.0 g, 54.0 mmol, 1.50 eq.) and then stirred for 11.5 h at 40° C. under N₂. LC-MS indicated E6 was consumed completely and a main peak with the desired mass was detected. The reaction solution was quenched with H₂O (100 mL) and extracted with ethyl acetate 150 mL (75.0 mL*2). The combined organic layers were washed with brine (30.0 mL*2) and concentrated under reduced pressure to give the crude product. The residue was purified by prep-HPLC (neutral condition, column: Phenomenex luna C18 (250*70 mm, 10 m); mobile phase: [water (NH₄HCO₃)-ACN]; gradient: 60%-90% B over 20 min). Compound E8 (14.0 g, 23.5 mmol, 65.5% yield) was obtained as a yellow oil. ¹H NMR (d₆-DMSO, 400 MHz): δ 8.37-8.31 (m, 2H), 8.19-8.14 (m, 2H), 8.00-7.95 (m, 1H), 7.56-7.45 (m, 2H), 7.29-7.10 (m, 3H), 6.92 (dd. J₁=7.6 Hz, J₂=4.8 Hz, 1H), 5.69-5.53 (m, 2H), 5.22 (dd, J₁=10.8, J₂=6.4 Hz, 1H), 4.83 (d, J=16.0 Hz, 1H), 4.44 (d, J=16.0 Hz, 1H), 3.52-3.40 (m, 2H), 2.56-2.52 (m, 3H), 1.96-1.58 (m, 3H), 0.77 (dd, J₁=8.6, J₂=7.4 Hz, 2H), −0.13 (s, 9H). LC-MS: EC14557-16-P1A3, (M+H)⁺=594.3.

### Step E1-8: Removal of Ns Protecting Group

To solution of compound E8 (14.0 g, 23.5 mmol, 1.00 eq.) in MeCN (80.0 mL) and DMF (40.0 mL) was added K₂CO₃ (9.78 g, 70.7 mmol, 3.00 eq.) and compound E9 (6.59 g, 59.8 mmol. 6.11 mL. 2.54 eq.), and then stirred at 50° C. for 2 h. LC-MS analysis indicated E8 was completely consumed and a main peak with the desired mass was detected. The reaction solution was quenched with H₂O (100 mL) and extracted with ethyl acetate (60.0 mL*2). The combined organic layers were washed with aqueous K₂CO₃ (50.0 mL*2), dried over Na₂SO₄, and concentrated under reduced pressure to give the crude product. The residue was purified by prep-HPLC (HCl condition, column: Phenomenex luna C18 (250*70 mm, 10 μm); mobile phase: [water (HCl)-ACN]; gradient: 17%-57% B over 25 min). Compound E10 (4.50 g, 11.0 mmol, 46.7% yield) was obtained as a brown oil. ¹H NMR (d₆-DMSO, 400 MHz): δ 8.37 (d, J=4.4 Hz, 1H), 7.62 (dd, J₁=8.0 Hz, J₂ 2.8 Hz, 1H), 7.50 (d, J=8.0 Hz, 1H), 7.29-7.13 (m, 3H), 5.70 (s, 2H), 4.27-4.08 (m, 2H), 3.86-3.73 (m, 1H), 3.59-3.45 (m, 2H), 3.23 (s, 1H), 2.82-2.69 (m, 2H), 2.15-2.06 (m, 1H), 1.95-1.85 (m, 1H), 1.77-1.60 (m, 2H), 0.87-0.75 (m, 2H), −0.12 (s, 9H).

### Step E1-9: Coupling of Fragments E10 and E11B

A solution of E10 (4.00 g, 9.79 mmol, 1.00 eq.) and E11B (2.02 g. 10.7 mmol, 1.10 eq.) in dichloromethane (40.0 mL) was stirred at 25° C. for 4 h, followed by addition of NaBH(OAc)₃ (3.11 g, 14.6 mmol, 1.50 eq.) and the reaction was then stirred at 25° C. for 2 h. TLC (plate 1, dichloromethane:methanol=10:1, R_{f}(R1)=0.20, R_{f}(P1)=0.30,) indicated E10 was consumed completely and a new spot was detected. The reaction solution was quenched with H₂O (30.0 mL) and extracted with dichloromethane (20.0 mL*2). The combined organic layers were washed with brine 20.0 mL, dried over Na₂SO₄, filtered, and concentrated under reduced pressure to afford E12 (4.00 g, crude) as a yellow oil, which was used for the next step directly.

### Step E1-10: Deprotection of E12

To a solution of E12 (4.00 g, 6.90 mmol, 1.00 eq.) in DCM (40.0 mL) was added TFA (76.7 g, 673 mmol, 50.0 mL, 97.5 eq.), and then stirred at 25° C. for 8 h. LC-MS indicated E12 was consumed completely and a main peak with the desired mass was detected. The residue was diluted with H₂O (50.0 mL), quenched by sat. aq. NaHCO₃ (60.0 mL) and extracted with DCM (50.0 mL*2). The combined organic layers were washed with brine (50.0 mL), dried over Na₂SO₄, filtered, and concentrated under reduced pressure to give a residue. The residue was purified by prep-HPLC (HCl condition, column: Phenomenex Luna C18 200*40 mm*10 μm; mobile phase: [water (HCl)-ACN]; gradient: 1%-23% B over 10 min). The product Mavorixafor (1.00 g, 2.76 mmol, 39.9% yield, 96.3% purity) was obtained as light-yellow solid, confirmed by ¹H NMR. LC-MS, HPLC, SFC, and OR. ¹H NMR (CDCl₃ 400 MHz): δ 8.60 (d, J=4.4 Hz, 1H), 7.60 (s, 2H), 7.43 (d, J=7.6 Hz, 1H), 7.23-7.17 (m, 2H), 7.15 (dd, J₁=7.6 Hz, J₂=4.8 Hz, 1H), 4.14-3.98 (m, 3H), 2.91-2.81 (m, 1H), 2.78-2.68 (m, 2H), 2.62-2.48 (m, 3H), 2.27-2.18 (m, 1H), 2.10-2.01 (m, 1H), 2.00-1.84 (m, 1H), 1.78-1.63.

### Example 2: Second Synthesis of Mavorixafor

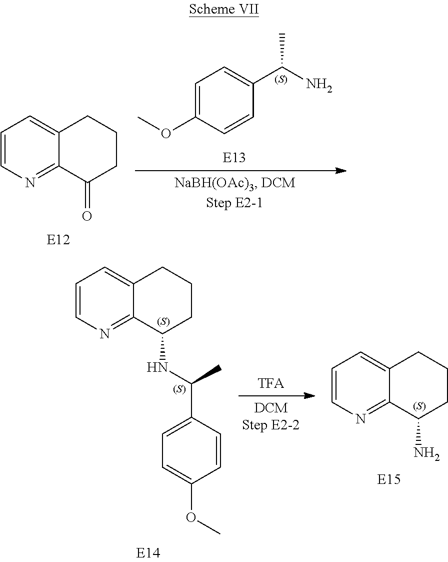

### Step E2-1: Synthesis of E14.

To a solution of compound E12 (20.0 g, 136 mmol, 1.00 eq) and compound E13 (20.6 g, 136 mmol, 20.1 mL, 1.00 eq) in DCM (400 mL) was added NaBH(OAc)₃ (51.8 g, 245 mmol, 1.80 eq) and the mixture was stirred at 25° C. for 12 hrs. LC-MS showed compound E12 was consumed completely and the desired mass was detected. The reaction mixture was diluted with water (500 mL) and extracted with DCM (300 mL*3). The organic layers were dried over Na₂SO₄, filtered, and concentrated under reduced pressure to give compound E14 (40.0 g, crude) as a yellow oil. ¹H NMR (400 MHz, CDCl₃): δ 8.43-8.42 (m, 1H), 7.48-7.36 (m, 3H), 7.13-7.07 (m, 1H), 6.94-6.88 (m, 2H), 4.39-4.37 (m, 1H), 3.92 (dd, J₁=4.0 Hz, J₂=4.0 Hz, 1H), 3.81 (s, 3H), 2.82-2.65 (m, 2H), 1.94-1.60 (m, 4H), 1.58-1.56 (d, J=8 Hz, 3H). LC-MS: (M+H)⁺:283.5.

### Step E2-2. Formation of Amine E15

To a solution of compound E14 (40.0 g, 142 mmol, 1.00 eq) in DCM (200 mL) was added TFA (307 g, 2.69 mol, 200 mL, 19.0 eq) and the mixture was stirred at 25° C. for 6 hrs. LC-MS showed compound E14 was consumed completely and the desired mass was detected. The reaction mixture was diluted with water (500 mL) and extracted with dichloromethane (500 mL). The pH was then adjusted to pH=12~13 with NaOH and extracted with dichloromethane (500 mL*3). The combined organic layers were dried over Na₂SO₄, filtered, and concentrated under reduced pressure to give a residue. The residue was purified by column chromatography (SiO₂, dichloromethane:methanol=1:0 to 0:1). TLC (dichloromethane:methanol=10:1. R_{f}(P1)=0.3). Compound E15 (14.0 g, 94.5 mmol. 66.7% yield) was obtained as a brown oil. ¹H NMR (400 MHz, CDCl₃): δ 8.42 (d, J=4.4 Hz, 1H), 7.37 (d, J=7.6 Hz, 1H), 7.06 (dd, J₁=7.2 Hz, J₂=4.4 Hz, 1H), 4.02 (t, J=7.2 Hz, 1H), 2.82-2.76 (m, 2H), 2.22-2.17 (m, 1H), 2.00-1.94 (m, 1H), 1.82-1.70 (m, 2H). LC-MS: (M+H)⁺: 149.1.

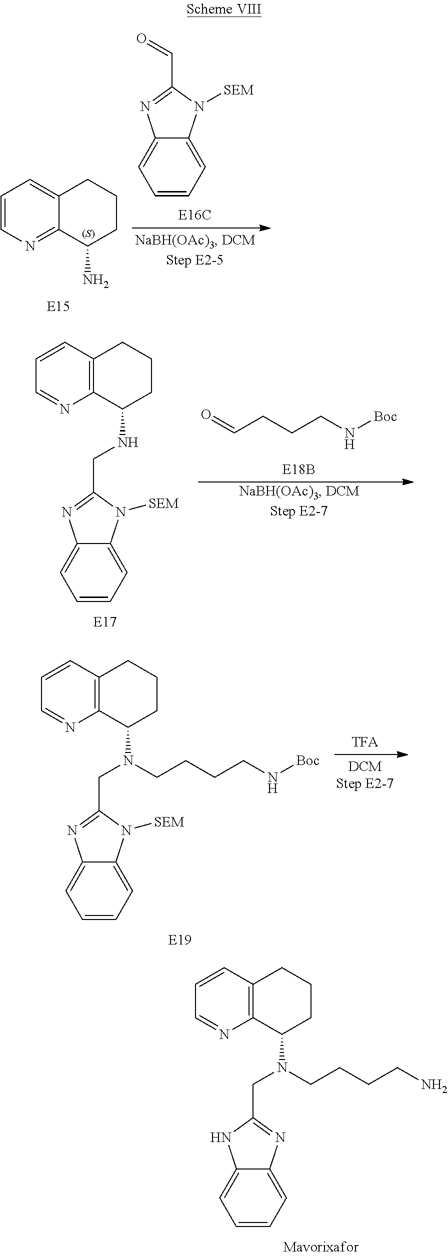

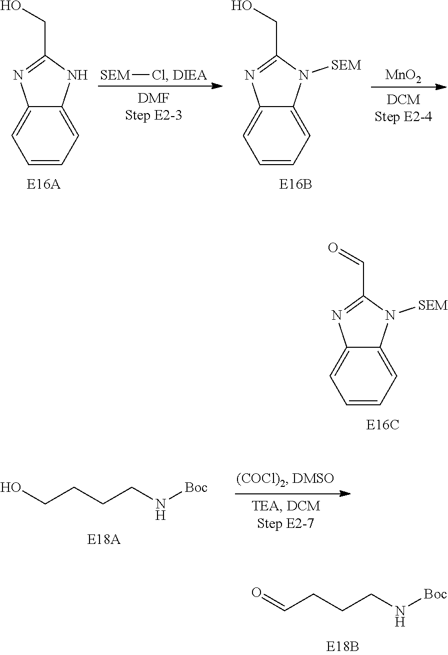

### Step E2-3. Protection of E16A

To a solution of compound E16A (22.0 g, 148 mmol, 1.00 eq) and DIEA (46.1 g, 356 mmol, 62.0 mL, 2.40 eq) in DMF (400 mL) was added SEM-Cl (28.5 g. 171 mmol. 30.0 mL, 1.15 eq) at 25° C. and the mixture was stirred at 60° C. for 2 hrs. LC-MS showed compound E16A was consumed completely and the desired mass was detected. The reaction mixture was diluted with water (300 mL), extracted with EtOAc (300 mL*3). The organic layers were dried over Na₂SO₄, filtered, and concentrated under reduced pressure to give a residue. The residue was purified by column chromatography (SiO₂, petroleum ether:ethyl acetate=1:0 to 1:3). TLC (petroleum ether:ethyl acetate=0:1, R_{f}(P1)=0.5). Compound E16B (25.0 g, 89.8 mmol, 60.5% yield) was obtained as a light-yellow oil. ¹H NMR: (400 MHz, CDCl₃): δ 7.74-7.66 (m, 1H), 7.44 (d. J=7.5 Hz, 1H), 7.33-7.26 (m, 2H), 5.60 (s, 2H), 4.95 (s, 2H), 4.54 (s, 1H), 3.56 (t, J=8.1 Hz, 2H), 0.90 (t, J=8.1 Hz, 2H), −0.06 (s, 9H). LC-MS: (M+H)⁺: 279.5.

### Step E2-4 Preparation of 6

To compound E16B (18.0 g. 64.6 mmol, 1.00 eq) in CH₂Cl₂ (200 mL) was added MnO₂ (28.1 g, 323 mmol, 5.00 eq) and the mixture was stirred at 25° C. for 12 hrs. LC-MS showed compound E16B was consumed completely and the desired mass was detected. The reaction mixture was filtered and concentrated under reduced pressure to give a residue. The residue was purified by column chromatography (SiO₂, petroleum ether:ethyl acetate=1:0 to 1:10). TLC (petroleum ether:ethyl acetate=5:1, R_{f}(P1)=0.3). Compound E16C (13.0 g, 47.0 mmol, 72.7% yield) was obtained as a light-yellow oil. ¹H NMR: (400 MHz, CDCl₃): δ 10.12 (s, 1H), 7.93 (d, J=8.1 Hz, 1H), 7.64 (d, J=8.3 Hz, 1H), 7.51-7.47 (m, 1H), 7.44-7.38 (m, 1H), 6.03 (s, 2H), 3.59-3.52 (m, 2H), 0.93-0.85 (m, 2H), −0.08 (s, 9H). LC-MS: (M+H)+: 276.9.

### Step E2-5: Coupling of E15 and E16C

To compound E15 (5.60 g, 37.8 mmol. 1.00 eq) and compound E16C (10.4 g, 37.8 mmol, 1.00 eq) in CH₂Cl₂ (100 mL) was added NaBH(OAc)₃ (12.0 g, 56.7 mmol, 1.50 eq), and the mixture was stirred at 25° C. for 6 hrs. LC-MS showed compound E15 was consumed completely and the desired mass was detected. The pH of the reaction mixture was adjusted to pH=7~8 with NaHCO₃ and extracted with CH₂Cl₂ (200 mL*3). The organic layers were dried over Na₂SO₄, filtered, and concentrated under reduced pressure to give compound E17 (19.0 g, crude) as a yellow oil. ¹H NMR: (400 MHz, CDCl₃): δ 8.40 (d, J=4.4 Hz, 1H), 7.81-7.73 (m, 1H), 7.50-7.44 (m, 1H), 7.39 (d, J=7.6 Hz, 1H), 7.30-7.28 (m, 2H), 7.10-7.07 (m, 1H), 5.80-5.65 (m, 2H) 0.4.43-4.26 (m, 2H), 3.98-3.89 (m, 1H), 3.58-3.54 (m, 2H), 2.86-2.73 (m, 2H), 2.25-2.15 (m, 1H), 2.07-1.97 (m, 1H), 1.90-1.73 (m, 2H), 0.94-0.90 (m, 2H), −0.05 (s, 9H). LC-MS: (M+H)⁺: 409.1.

### Step E2-6. Oxidation of E18A

To a solution of (COCl)₂ (6.71 g, 52.8 mmol, 4.63 mL, 1.00 eq) in CH₂Cl₂ (60.0 mL) was added DMSO (4.95 g, 63.4 mmol, 4.95 mL, 1.20 eq) in CH₂Cl₂ (10 mL) dropwise at −78° C. and stirred for 15 min under N₂. Compound E18A (10.0 g, 52.8 mmol, 1.00 eq) in CH₂Cl₂ (20.0 mL) was then added dropwise at −78° C. and stirred for 20 min. Finally, TEA (16.0 g, 158 mmol, 22.0 mL, 3.00 eq) was added dropwise at −78° C. and the mixture was warmed up to 20° C. and stirred for 30 min under N₂. TLC showed compound E18A was consumed completely (petroleum ether:ethyl acetate=1:1, R_{f}(R1)=0.2, R_{f}(P1)=0.6). The reaction mixture was diluted with water (200 mL) and extracted with CH₂Cl₂ (200 mL*3), the organic layers dried over Na₂SO₄, filtered, and concentrated under reduced pressure to give a residue. Compound E18B (9.00 g. crude) was obtained as a light-yellow oil.

Step E2-7. Coupling of compounds E17 and E18B

Compound E17 (18.6 g, 45.4 mmol, 1.00 eq) and compound E18B (8.50 g. 45.4 mmol, 1.00 eq) in CH₂Cl₂ (150 mL) were stirred at 25° C. for 4 hrs, followed by addition of NaBH(OAc)₃ (14.4 g, 68.1 mmol, 1.50 eq) and stirring at 25° C. for 2 hrs. LC-MS showed compound 7 was consumed completely and the desired mass was detected. The pH of the reaction mixture was adjusted to pH=7~8 with NaHCO₃ and extracted with CH₂Cl₂ (200 mL*3). The organic layers were dried over Na₂SO₄, filtered, and concentrated under reduced pressure to give compound E19 (25.0 g, 43.1 mmol, 94.97% yield) as a yellow oil. Then the product was purified by Prep-SFC (column: REGIS (s, s) WHELK-O1 (250 mm*50 mm, 10 um); mobile phase: [CO₂-MeOH (0.1% NH₃H₂O)]; B %: 35%, isocratic elution mode) to afford compound E19 (16 g, 25.04 mmol. 58.08% yield, 90.75% purity) as a yellow oil. ¹H NMR (400 MHz, CDCl₃): δ 8.48 (d, J=4.0 Hz, 1H), 7.72-7.70 (m, 1H), 7.45-7.43 (m, 1H), 7.31-7.21 (m, 3H), 7.23-7.01 (m, 1H), 6.02 (d, J=11.2 Hz, 1H), 5.79 (d, J=11.2 Hz, 1H), 4.93 (brs, 1H), 4.23-4.06 (m, 2H), 3.41-3.35 (m, 2H), 3.00-2.95 (m, 2H), 2.84-2.77 (m, 1H), 2.69-2.64 (m, 3H), 2.15-2.09 (m, 1H), 2.02-1.98 (m, 2H), 1.69-1.58 (m, 2H), 1.50-1.45 (m, 4H), 1.43 (s, 9H), 6.02 (t, J=16.4 Hz. 2H), −0.10 (s, 9H). LC-MS: (M+H)⁺: 580.9.

### Step E2-7. Deprotection of E19

To compound E19 (5.00 g, 7.83 mmol, 1.00 eq) in CH₂Cl₂ (50.0 mL) was added TFA (76.7 g. 673 mmol. 50.0 mL, 86.0 eq) and the mixture was stirred at 25° C. for 8 hrs. LC-MS showed compound E19 was consumed completely and the desired mass was detected. The pH of the reaction mixture was adjusted to pH=7~8 with NaHCO₃ and extracted with CH₂Cl₂ (500 mL*3). The combined organic layers were dried over Na₂SO₄, filtered, and concentrated under reduced pressure to give the crude product. The crude product was purified by Prep-HPLC (column: Phenomenex luna Cis (250*70 mm, 10 um); mobile phase: [water (HCl)-ACN]; gradient: 0%-20% B over 20 min). Mavorixafor (1.30 g, 7.15 mmol) was obtained as a light-yellow solid, confirmed by ¹H NMR, LC-MS, SFC, and OR. ¹H NMR: (400 MHz, CDCl₃): δ 8.60 (d, J=4.0 Hz, 1H), 7.60 (s, 2H), 7.43 (d, J=7.6 Hz, 1H), 7.24-7.10 (m, 3H), 4.13-4.01 (m, 3H), 2.91-2.81 (m, 1H), 2.79-2.68 (m, 2H), 2.63-2.47 (m, 3H), 2.27-2.17 (m, 1H), 2.10-2.01 (m, 1H), 1.98-1.86 (m, 1H), 1.77-1.64 (m, 1H), 1.46-1.36 (m, 4H). LC-MS: (M+H)+: 350.2

### Example 3: Third Synthesis of Mavorixafor

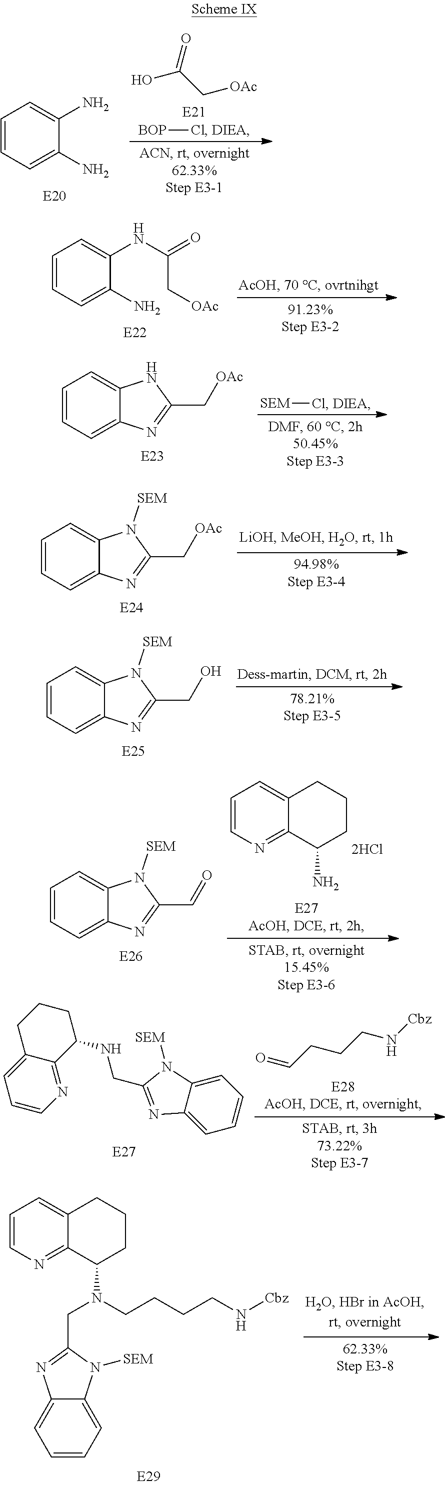

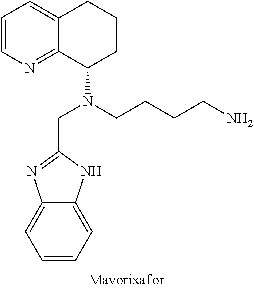 [SMILES: NCCCCN(Cc1nc2ccccc2[nH]1)[C@H]1CCCc2cccnc21]

### Step E3-1. Preparation of E22

DIEA (24.16 mL, 138.703 mmol, 1.5 equiv.) and bis(2-oxo-1,3-oxazolidin-3-yl)phosphinoyl chloride (35.3 g, 138.703 mmol, 1.5 equiv.) were added to a stirred solution of E20 (10.0 g, 92.469 mmol, 1 equiv.) and E21 (10.9 g, 92.469 mmol, 1 equiv.) in ACN (150 mL) at room temperature. The resulting mixture was stirred overnight at room temperature. The resulting mixture was concentrated under reduced pressure. The residue was purified by silica gel column chromatography and eluted with CH₂Cl₂/MeOH (9:1) to afford E22 (12.0 g, 62.33%) as an off-white solid. MS (ESI): 209.3 [M+H]+.

### Step E3-2. Cyclization of E22

A solution of E22 (12.0 g, 57.632 mmol, 1 equiv.) in AcOH (120 mL) was stirred for overnight at 70° C. The resulting mixture was concentrated under reduced pressure and diluted with water (500 mL). The mixture was basified to pH 8 with saturated NaHCO₃ (aq.). The resulting mixture was extracted with EtOAc (3×500 mL). The combined organic layers were washed with NaHCO₃ (aq.) (3×500 mL), dried over anhydrous Na₂SO₄, filtered, and concentrated under reduced pressure. This resulted in E23 (10.0 g, 91.23%) as a light-yellow solid. MS (ESI): 191.4 [M+H]+.

### Step E3-3. SEM Protection of E23

To a stirred solution of E23 (10.0 g, 52.576 mmol, 1 equiv.) and DIEA (17.0 g, 131.440 mmol, 2.5 equiv.) in DMF (150 mL) was added [2-(chloromethoxy)ethyl]trimethylsilane (13.2 g, 78.864 mmol, 1.5 equiv.) at 0° C. under a nitrogen atmosphere. The resulting mixture was stirred for 2 h at 60° C. under a nitrogen atmosphere. The resulting mixture was diluted with water (500 mL) and extracted with EtOAc (3×500 mL). The combined organic layers were washed with brine (3×500 mL), dried over anhydrous Na₂SO₄, and filtered. The filtrate was concentrated under reduced pressure. The residue was purified by silica gel column chromatography, eluted with PE/EA (2:1) to afford E24 (8.5 g, 50.45%) as a light yellow solid. MS (ESI): 321.4 [M+H]+.

### Step E3-4. Acetate Cleavage of E24

A solution E24 (10.3 g, 32.141 mmol, 1 equiv.) and LiOH (1.5 g, 64.282 mmol, 2 equiv.) in MeOH (200 mL) and H₂O (66 mL) was stirred for 1 h at room temperature. The resulting mixture was diluted with water (500 mL) and extracted with EtOAc (3×500 mL). The combined organic layers were washed with brine (3×500 mL), dried over anhydrous Na₂SO₄, and filtered. The filtrate was concentrated under reduced pressure. This resulted in E25 (8.5 g, 94.98%) as a light yellow oil. MS (ESI): 279.1 [M+H]+.

### Step E3-5. Oxidation of Alcohol E25

To a stirred solution of E25 (8.5 g, 30.529 mmol, 1 equiv.) in DCM (200 mL) was added Dess-martin (32.4 g. 76.323 mmol, 2.5 equiv.) in portions at 0° C. under a nitrogen atmosphere. The resulting mixture was stirred for 2 h at room temperature under a nitrogen atmosphere. The resulting mixture was diluted with water (500 mL) and extracted with CH₂Cl₂ (3×500 mL). The combined organic layers were washed with NaHCO₃ (aq.) (3×500 mL), dried over anhydrous Na₂SO₄, and filtered. The filtrate was concentrated under reduced pressure. The residue was purified by reversed-phase flash chromatography with the following conditions: stationary phase: C18 silica gel: mobile phase: MeCN in Water (0.1% NH₃·H₂O), 0% to 100% gradient in 15 min: detector: UV 254 nm. E26 (6.6 g, 78.21%) was isolated as a yellow oil. MS (ESI): 277.1 [M+H]+.

### Step E3-6. Coupling of Fragments E26 and E27

To a stirred solution of E26 (3.8 g. 13.567 mmol, 1 equiv.) and E27 (3.0 g. 13.567 mmol, 1.00 equiv.) in DCE (60 mL) was added AcOH (2.4 g, 40.701 mmol, 3 equiv.) at room temperature. The resulting mixture was stirred for 2 h at room temperature. To the above mixture was added STAB (8.6 g, 40.701 mmol, 3 equiv.) in portions over 5 min at 0° C. The resulting mixture was then stirred for overnight at room temperature. The resulting mixture was diluted with water (300 mL) and extracted with CH₂Cl₂ (3×300 mL). The combined organic layers were washed with brine (3×300 mL), dried over anhydrous Na₂SO₄, and filtered. The filtrate was concentrated under reduced pressure. The residue was purified by reversed-phase flash chromatography with the following conditions: stationary phase: C18 silica gel; mobile phase: MeCN in Water (0.1% NH₃·H₂O), 0% to 100% gradient in 15 min; detector: UV 254 nm. E27 (850.0 mg, 15.45%) was isolated as a yellow oil. MS (ESI): 409.6 [M+H]⁺.

### Step E3-7. Coupling of Fragments E27 and E28

To a stirred solution of E27 (2.0 g, 4.895 mmol, 1 equiv.) and E28 (1.6 g, 7.342 mmol, 1.5 equiv.) in DCE (40 mL) was added AcOH (881.8 mg, 14.685 mmol. 3 equiv.) at room temperature. The resulting mixture was stirred overnight at room temperature. To the above mixture STAB (5.2 g, 24.475 mmol, 5 equiv.) was added in portions over 3 min at 0° C. The resulting mixture was stirred for an additional 3 h at room temperature. The resulting mixture was diluted with water (300 mL) and extracted with CH₂Cl₂ (3×300 mL). The combined organic layers were washed with brine (3×300 mL), dried over anhydrous Na₂SO₄, and filtered. The filtrate was concentrated under reduced pressure. The residue was purified by reversed-phase flash chromatography with the following conditions: stationary phase: C18 silica gel: mobile phase: MeCN in Water (0.1% NH₃·H₂O), 0% to 100% gradient in 15 min; detector: UV 254 nm. E29 (2.2 g, 73.22%) was isolated as a light yellow oil. MS (ESI): 614.9 [M+H]⁺.

### Step E3-8. Deprotection of E29

A solution of E29 (3.1 g, 5.050 mmol, 1 equiv.) in HBr in AcOH (60 mL, 33%) and H₂O (10 mL) was stirred overnight at room temperature. The resulting mixture was concentrated under reduced pressure and diluted with water (300 mL). The mixture was basified to pH 8 with saturated NaHCO₃ (aq.). The resulting mixture was extracted with CH₃Cl:IPA=3:1 (3×300 mL). The combined organic layers were washed with NaHCO₃ (aq.) (3×300 mL), dried over anhydrous Na₂SO₄, and filtered. The filtrate was concentrated under reduced pressure. The residue was purified by reversed-phase flash chromatography with the following conditions: stationary phase: C18 silica gel; mobile phase: MeCN in Water (10 mmol/L NH₄HCO₃), 0% to 100% gradient in 15 min; detector: UV 254 nm to afford Mavorixafor (1.1 g, 62.33%) as an off-white solid. Column: Zorbax Bonus RP 150*4.6 mm, 3.5 um. Rt (min): 13.45; Area %—96.1. ¹H NMR (400 MHz, d₄-Methanol): δ 8.58-8.47 (m, 1H), 7.63-7.48 (m, 3H), 7.30-7.13 (m, 3H), 4.22-3.93 (m, 3H), 2.87 (ddd, J=16.3, 10.7, 5.0 Hz, 1H), 2.75 (dt, J=16.7, 4.5 Hz, 1H), 2.67-2.57 (m, 1H), 2.56-2.38 (m, 3H), 2.29-2.18 (m, 1H), 2.04 (ddd. J=17.0, 10.4, 6.3 Hz, 1H), 1.98-1.87 (m, 1H), 1.69 (tdt, J=13.3, 7.5, 2.8 Hz, 1H), 1.38 (qt, J=8.1, 5.2 Hz, 4H). LCMS: 350.2 (M+H).

### Example 4: Fourth Synthesis of Mavorixafor

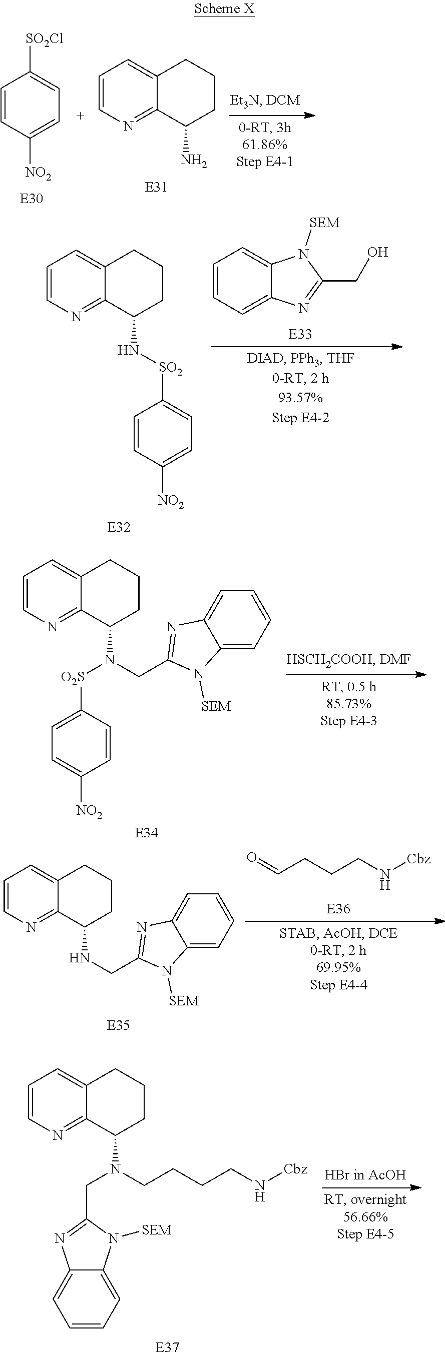

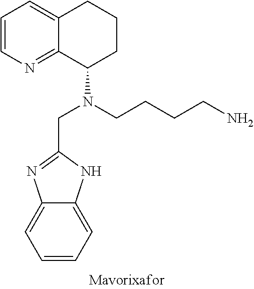 [SMILES: NCCCCN(Cc1nc2ccccc2[nH]1)[C@H]1CCCc2cccnc21]

### Step E4-1: Protection of E31

To a stirred mixture of E31 (6 g, 40.483 mmol, 1.00 equiv.) and Et₃N (12.29 g, 121.449 mmol. 3 equiv.) in DCM (50 mL) was added E30 (17.94 g. 80.966 mmol, 2 equiv.) in portions at 0° C. under a nitrogen atmosphere. The resulting mixture was stirred for additional 3 h at room temperature. The reaction was quenched by the addition of water (10 mL) at 0° C. The resulting mixture was extracted with CH₂Cl₂ (3×100 mL). The combined organic layers were washed with water (3×50 mL), dried over anhydrous Na₂SO₄. After filtration, the filtrate was concentrated under reduced pressure. The residue was purified by silica gel column chromatography, eluted with CH₂Cl₂/PE (1:1) to afford E32 (8.5 g, 61.86%) as yellow solid. MS (ESI): 334.1 [M+H]⁺.

### Step E4-2: Coupling of Fragments E32 and E33.

To a stirred mixture of E33 (3.76 g, 13.498 mmol, 1.5 equiv.) and E32 (3 g, 8.999 mmol, 1.00 equiv.) in THF (20 mL, 1.800 mmol) was added DIAD (5.46 g, 26.997 mmol, 3 equiv.) dropwise at 0° C. under a nitrogen atmosphere. The resulting mixture was stirred for additional 3 h at room temperature. The resulting mixture was extracted with EtOAc (3×100 mL). The combined organic layers were washed with water (2×50 mL), dried over anhydrous Na₂SO₄, and filtered. The filtrate was concentrated under reduced pressure. The residue was purified by silica gel column chromatography and eluted with CH₂Cl₂/MeOH (10:1) to afford E34 (5 g. 93.57%) as a yellow solid. MS (ESI): 594.8 [M+H]⁺.

### Step E4-3. E34 Amine Deprotection

To a stirred mixture of E34 (5.0 g, 8.421 mmol, 1 equiv.) and DBU (12.82 g, 84.210 mmol, 10 equiv.) in DMF (30 mL) was added 2-sulfanylacetic acid (3.88 g. 42.105 mmol, 5 equiv.) dropwise at 0° C. under a nitrogen atmosphere. The resulting mixture was stirred for an additional 20 min at room temperature. The resulting mixture was diluted with water (50 mL). The mixture was extracted with EtOAc (3×100 mL). The combined organic layers were washed with water (3×30 mL), dried over anhydrous Na₂SO₄, and filtered. The filtrate was concentrated under reduced pressure. The residue was purified by reversed-phase flash chromatography with the following conditions: stationary phase: C18 silica gel; mobile phase: MeCN in Water (0.1% FA), 40% to 90% gradient in 10 min; detector: UV 254 nm. E35 (2.95 g, 85.73%) was isolated as an orange oil. MS (ESI): 409.6 [M+H]⁺.

### Step E4-4. Coupling of E35 and E36

A solution of (8S)—N-[(1-{[2-(trimethylsilyl)ethoxy]methyl}-1,3-benzodiazol-2-yl)methyl]-5,6,7,8-tetrahydroquinolin-8-amine (2.95 g, 7.219 mmol, 1 equiv.) and solution of benzyl N-(4-oxobutyl)carbamate (4.79 g. 21.657 mmol. 3.00 equiv.) in DCE (40 mL) was treated with AcOH (2.17 g, 36.095 mmol, 5 equiv.) for 12 h at room temperature under a nitrogen atmosphere, followed by the addition of STAB (4.59 g, 21.657 mmol, 3 equiv.) in portions at 0° C. The resulting mixture was stirred for additional 5 h at room temperature. The resulting mixture was diluted with water (30 mL). The resulting mixture was extracted with EtOAc (3×100 mL). The combined organic layers were washed with water (2×40 mL), dried over anhydrous Na₂SO₄, and filtered. The filtrate was concentrated under reduced pressure. The residue was purified by reversed-phase flash chromatography with the following conditions: stationary phase: C18 silica gel; mobile phase: MeCN in Water (0.1% FA), 40% to 90% gradient in 10 min: detector: UV 254 nm. E37 (3.1 g, 69.95%) was isolated as an orange oil. MS (ESI): 614.9 [M+H]⁺.

### Step E4-5. Deprotection of E37

To a stirred mixture of E37 (3.1 g, 5.050 mmol, 1 equiv.) in H2O (10 mL) was added HBr in AcOH (60 mL. 33%) dropwise at 0° C. under a nitrogen atmosphere. The resulting mixture was stirred for an additional 12 h at room temperature. The resulting mixture was concentrated under reduced pressure and diluted with water (250 mL). The mixture was basified to pH 8 with saturated NaHCO₃ (aq.). The resulting mixture was extracted with CH₃Cl:IPA=3:1 (3×250 mL). The combined organic layers were washed with brine (3×500 mL), dried over anhydrous Na₂SO₄, and filtered. The filtrate was concentrated under reduced pressure. The residue was purified by reversed-phase flash chromatography with the following conditions: stationary phase: C18 silica gel; mobile phase: MeCN in Water (10 mmol/L NH₄HCO₃), 40% to 100% gradient in 10 min; detector: UV 254 nm. This resulted in Mavorixafor (1.0 g, 56.66%) as an off-white solid. ¹H NMR (400 MHz, d₄-Methanol): δ 8.50 (d, J=4.8 Hz, 1H), 7.59-7.49 (m, 3H), 7.27-7.16 (m, 3H), 4.13 (dd, J=9.8, 6.1 Hz, 1H), 4.07 (d, J=15.5 Hz, 1H), 3.99 (d, J=15.4 Hz, 1H), 2.93-2.81 (m, 1H), 2.80-2.71 (m, 1H), 2.69-2.60 (m, 1H), 2.60-2.51 (m, 2H), 2.45 (dt, J=12.9, 7.1 Hz, 1H), 2.28-2.20 (m, 1H), 2.09-1.88 (m, 2H), 1.78-1.65 (m, 1H), 1.48-1.33 (m, 4H). MS (ESI): 350.1 [M+H]⁺.

### Example 5: Fifth Synthesis of Mavorixafor

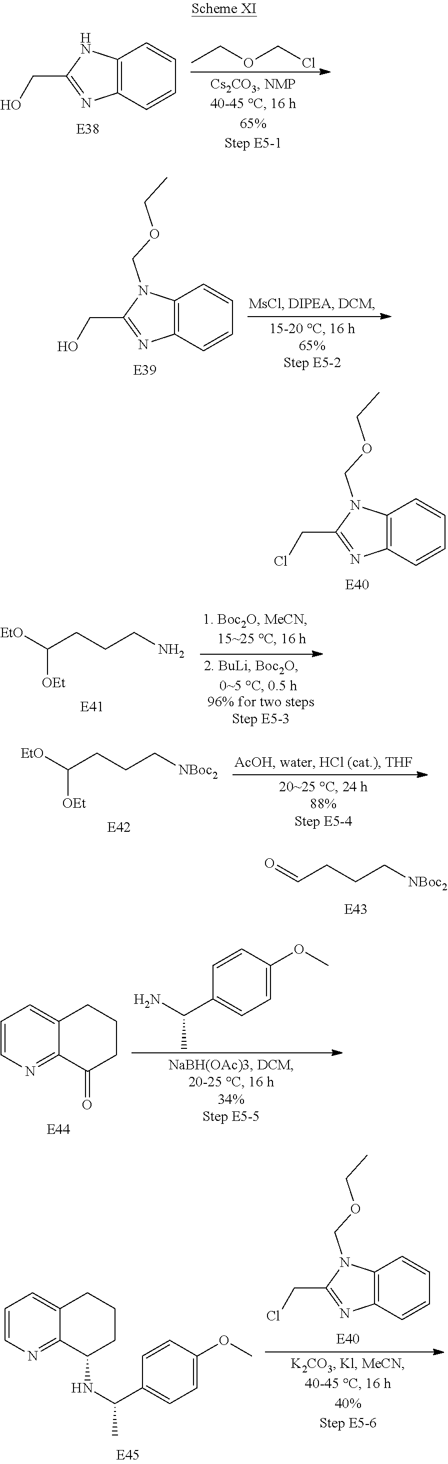

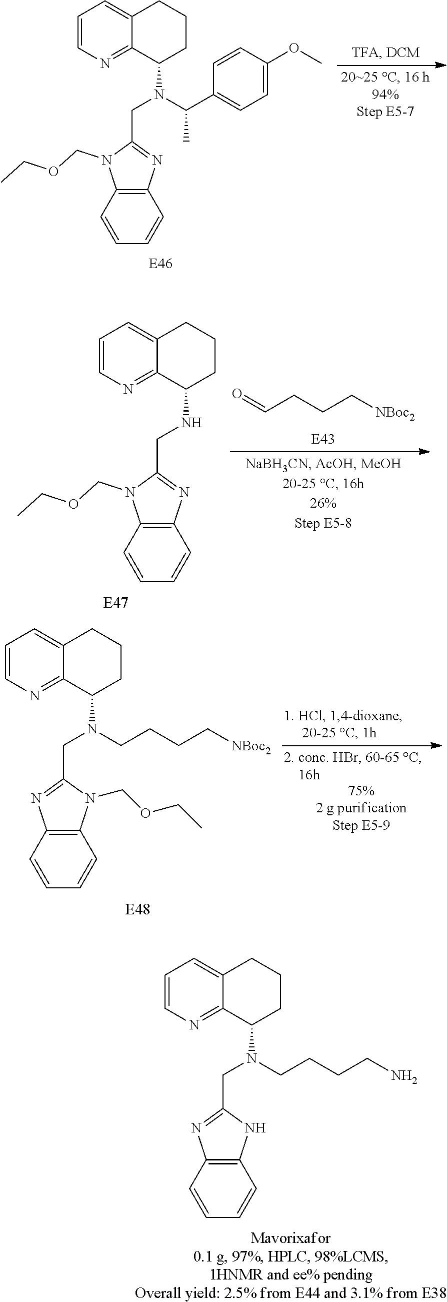

### Step E5-1. Protection of E38

To a solution of (1H-benzo[d]imidazol-2-yl)methanol (E38, 15 g, 101.24 mmol) in NMP (150 mL) was added Cs₂CO₃ (46.18 g, 141.73 mmol) and (chloromethoxy)ethane (EOMCI, 12.92 g, 136.67 mmol) at 20-25° C., then the mixture was stirred at 20-25° C. for 1 h and then at 40-45° C. for 16 h. At this point, 84% conversion to product and 15% of E38 remains according to LCMS. The mixture was poured into EtOAc (450 mL) and water (450 mL). The organic phase was collected and the aqueous phase was extracted with the EtOAc (300 mL×1). The combined organic phase was concentrated in vacuum to give a residue, and the residue was purified by column chromatography (DCM:MeOH=50:1) to give (1-(ethoxymethyl)-1H-benzo[d]imidazol-2-yl)methanol (E39, 13.5 g, 65%) as a yellow oil.

### Step E5-2. Halogenation of E39

To a solution of (1-(ethoxymethyl)-1H-benzo[d]imidazol-2-yl)methanol (E39, 30.0 g, 145.46 mmol) and DIPEA (28.20 g, 218.19 mmol) in DCM (300 mL) was added methanesulfonyl chloride (24.99 g, 218.19 mmol) at 0-5° C. Then, the mixture was stirred at 15-20° C. for 16 h. After completion of the reaction as indicated by LCMS, the mixture was washed with water (450 mL×1), aq. NaHCO₃ (150 mL×1) and brine (150 mL×1). The organic phase was dried over Na₂SO₄ and concentrated to give crude 2-(chloromethyl)-1-(ethoxymethyl)-1H-benzo[d]imidazole (E40, 13.5 g, 65%) as a yellow oil.

### Step E5-3. Protection of E41

To a solution of 4,4-diethoxybutan-1-amine (E41, 20 g, 124.22 mmol) in MeCN (200 mL) was added Boc₂O (27.08 g. 124.22 mmol) at 20-25° C., and then the mixture was stirred at 20-25° C. for 16 h. After completion of the reaction as indicated by TLC and GCMS, the mixture was poured into water (200 mL) and extracted with MTBE (200 mL×3). The combined organic phase was washed with brine (100 mL×2), dried over Na₂SO₄, filtered, and concentrated in vacuum to give tert-butyl (4,4-diethoxybutyl)carbamate (33 g, 90%) as yellow oil. To a solution of tert-butyl (4,4-diethoxybutyl)carbamate (28 g, 107.28 mmol) in THF (280 mL) was added nBuLi (64.4 ml, 160.92 mmol) at −5-0° C. under N2 protection. After stirring for extra 20 min Boc₂O (42 g, 160.92 mmol) wads added dropwise at −5-0° C. and the mixture was stirred at −5-0° C. for 0.5 h. After completion of the reaction indicated by TLC and GCMS, the reaction was quenched by adding aq. NH₄Cl (10 wt %, 30 mL) and then the mixture was poured into water (200 mL) and extracted with MTBE (200 mL×3). The combined organic phase was washed with brine (100 mL×2), dried over Na₂SO₄, filtered, and concentrated in vacuum to give tert-butyl (tert-butoxycarbonyl)(4,4-diethoxybutyl)carbamate (E42, 41 g, 106%) as a yellow oil.

### Step E5-4. Preparation of E43

To a solution of tert-butyl (tert-butoxycarbonyl)(4,4-diethoxybutyl)carbamate (E42, 20 g, 55.4 mmol) in THF (200 mL) was added AcOH (80 ml) and H₂O (40 ml) at 20-25° C., followed by dropwise addition of conc. HCl (12 M, 1 g, 5.54 mmol). The mixture was stirred at 20-25° C. for 16 h. After completion of the reaction as indicated by TLC and GCMS, the mixture was extracted with a mixed solution of MTBE/PE (v/v=1:1, 300 mL). The organic phase was washed with aq. sat. NaHCO₃ (200 mL×1), brine (200 mL×1), dried over Na₂SO₄, filtered and concentrated to give tert-butyl (tert-butoxycarbonyl)(4-oxobutyl)carbamate (E43, 14 g, 88%) as a yellow oil.

### Step E5-5. Reductive Amination of E44

To a solution of 6,7-dihydroquinolin-8 (5H)-one (E44, 20 g, 135.89 mmol) in DCM (500 mL) was added (S)-1-(4-methoxyphenyl)ethanamine (20.55 g, 135.89 mmol) and NaBH(OAc)₃ (57.60 g, 271.78 mmol) at 20-25°. The mixture was stirred at 20-25° C. for 16 h. After completion of the reaction as indicated by LCMS, the mixture was washed with aq. NaHCO₃ (200 mL×1), brine (200 mL×1), dried over Na₂SO₄, filtered, and concentrated in vacuum to give a residue. The residue was purified by column chromatography (DCM:MeOH=100:1) to give a brown product, which was then recrystallized with toluene (60 mL) at 0-5° C. for 2 h. The solid was then collected by filtration and dried under reduced pressure by oil pump at 20-25° C. to give (S)—N—((S)-1-(4-methoxyphenyl)ethyl)-5,6,7,8-tetrahydroquinolin-8-amine (E45, 13 g, 34%) as an off-white solid with 98% de.

### Step E5-6. Alkylation of E45

To a solution of (S)—N—((S)-1-(4-methoxyphenyl)ethyl)-5,6,7,8-tetrahydroquinolin-8-amine (E45, 6.0 g. 21.25 mmol) in MeCN (60 mL) was added 2-(chloromethyl)-1-(ethoxymethyl)-1H-benzo[d]imidazole (E40, 5.73 g, 25.50 mmol), K₂CO₃ (5.29 g, 38.25 mmol) and KI (317 mg, 1.91 mmol). The mixture was stirred at 40-45° C. for 16 h, at which point LCMS indicated 61% product formation with 33% of E45 remaining. The mixture was poured into water (200 mL) and extracted with EA (200 mL×2). The combined organic phase was washed with brine (150 mL×2), dried over Na₂SO₄, filtered, and concentrated in vacuum to give a residue, which was purified by column chromatography (PE:EA=6:1 to EA) to give (S)—N-((1-(ethoxymethyl)-1H-benzo[d]imidazol-2-yl)methyl)-N—((S)-1-(4-methoxyphenyl)ethyl)-5,6,7,8-tetrahydroquinolin-8-amine (E46, 4.0 g, 40%) as a yellow oil.

### Step E5-7. Deprotection of E46

To a solution of (S)—N-((1-(ethoxymethyl)-1H-benzo[d]imidazol-2-yl)methyl)-N—((S)-1-(4-methoxyphenyl)ethyl)-5,6,7,8-tetrahydroquinolin-8-amine (E46, 3.5 g, 8.50 mmol) in dry DCM (35 mL) was added TFA (10.5 mL) at 20-25° C., then the mixture was stirred at 20-25° C. for 16 h. After completion of the reaction as indicated by LCMS, the mixture was concentrated in vacuum, diluted with water (30 mL), adjusted to pH=9 by aq. sat. NaHCO₃ solution and extracted with DCM (150 mL×3). The organic layer was washed with brine (150 mL×1), dried over Na₂SO₄, filtered and concentrated in vacuum to give a residue, which was purified by column chromatography (DCM:MeOH=50:1) to give (S)—N-((1-(ethoxymethyl)-1H-benzo[d]imidazol-2-yl)methyl)-5,6,7,8-tetrahydroquinolin-8-amine (E47, 2.7 g, 94%) as a brown oil.

### Step E5-8. Reductive Amination of E47

To a solution of (S)—N-((1-(ethoxymethyl)-1H-benzo[d]imidazol-2-yl)methyl)-5,6,7,8-tetrahydroquinolin-8-amine (E47, 3.9 g, 11.61 mmol) in MeOH (40 mL) was added tert-butyl (tert-butoxycarbonyl)(4-oxobutyl)carbamate (E43, 7.3 g, 25.54 mmol), AcOH (14 mg, 0.23 mmol) and NaBH₃CN (0.73 g, 11.61 mmol), then the mixture was stirred at 20-25° C. for 16 h under N₂ protection. At this point, LCMS indicated that 72% of the product had formed with ~4% of E47 remaining. The mixture was poured into water (40 mL) and extracted with EA (40 mL×2). The combined organic phase was washed with aq. NaHCO₃ (40 mL×1), brine (20 mL×2), dried over Na₂SO₄, filtered, and concentrated under vacuum to give a residue, which was purified by column chromatography (DCM:MeOH=100:1) to give 3.8 g of product with 83% LCMS purity. The product (with 83% LCMS purity) was dissolved with MTBE (80 mL), washed with aq. citric acid (120 mg in 20 mL of water, ×1), aq. sat. NaHCO₃ (20 mL×1), dried over Na₂SO₄, filtered and concentrated in vacuum to give tert-butyl (S)-(tert-butoxycarbonyl)(4-(((1-(ethoxymethyl)-1H-benzo[d]imidazol-2-yl)methyl)(5,6,7,8-tetrahydroquinolin-8-yl)amino)butyl)-carbamate (E48, 3.2 g, 26%) as a brown oil.

### Step E5-9. Deprotection of E48

To a solution of tert-butyl (S)-(tert-butoxycarbonyl)(4-(((1-(ethoxymethyl)-1H-benzo[d]imidazol-2-yl)methyl)(5,6,7,8-tetrahydroquinolin-8-yl)amino)butyl)carbamate (E48, 2.0 g, 3.29 mmol) in 1,4-dioxane (8 mL), was added 4 N HCl in 1,4-dioxane (8 mL) under N₂ protection at 20-25° C., and the mixture was stirred at 20-25° C. for 1 h under N2 protection. After completion of the reaction as indicated by LCMS, the organic phase was decanted and the semisolid was dissolved with conc. HBr (44 wt %, 8 mL) and concentrated to give a crude salt. The crude salt was dissolved with conc. HBr (44 wt %, 20 mL) and then the mixture was stirred at 60-65° C. for 16 h under N₂ protection. After completion of the reaction as indicated by LCMS, the mixture was poured into water (40 mL), washed with toluene (20 mL×3), and concentrated in vacuum to give a brown residue. The residue was dissolved in water (4 mL) and then added dropwise into aq. NH₃ (10 wt %, 18 mL) at 0-5° C. After stirring for an extra 20 min at 0-5° C., the aqueous phase was decanted and the semisolid was washed with water (2 mL×2), dried under high vacuum (oil pump).

### Example 6: Sixth Synthesis of Mavorixafor

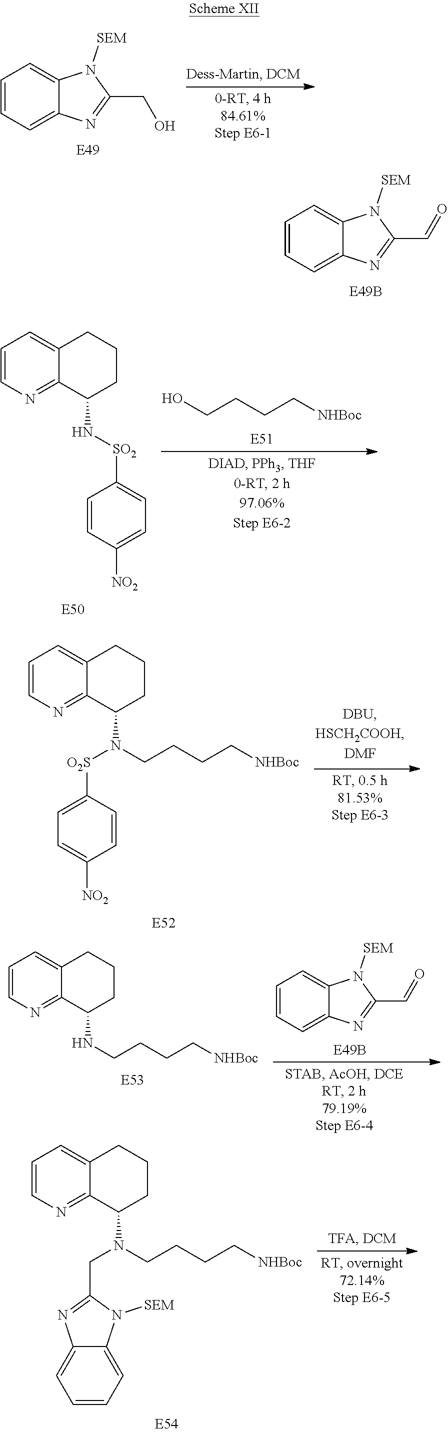

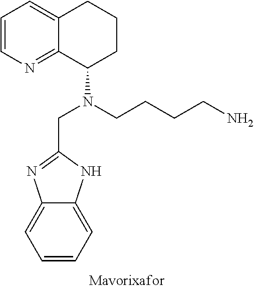 [SMILES: NCCCCN(Cc1nc2ccccc2[nH]1)[C@H]1CCCc2cccnc21]

### Step E6-1: Preparation of Aldehyde E49B

To a stirred solution/mixture of (1-{[2-(trimethylsilyl)ethoxy]methyl}-1,3-benzodiazol-2-yl)methanol (5 g. 17.958 mmol, 1 equiv.) in DCM (100 mL) was added Dess-Martin (22.85 g, 53.874 mmol, 3 equiv.) in portions at 0° C. under a nitrogen atmosphere. The resulting mixture was stirred for 4 h at room temperature under a nitrogen atmosphere. The resulting mixture was diluted with DCM (50 mL). The combined organic layers were washed with NaHCO₃ (4×50 mL), dried over anhydrous Na₂SO₄ and filtered. The filtrate was concentrated under reduced pressure. The residue was purified by reversed-phase flash chromatography with the following conditions: column: C18 silica gel; mobile phase: MeCN in Water (0.1% NH₃·H₂O), 0% to 87% gradient in 15 min; detector: UV 254 nm to afford 1-{[2-(trimethylsilyl)ethoxy]methyl}-1,3-benzodiazole-2-carbaldehyde (4.2 g, 84.61%) as a colorless oil. MS (ESI): 277.0 [M+H]+.

### Step E6-2: Preparation of Compound E52

To a stirred solution of 4-nitro-N-[(8S)-5,6,7,8-tetrahydroquinolin-8-yl]benzenesulfonamide (2.11 g, 6.329 mmol, 1 equiv.) and tert-butyl N-(4-hydroxybutyl)carbamate (2.40 g, 12.658 mmol, 2 equiv.), PPh₃ (4.98 g, 18.987 mmol, 3 equiv.) in THF (40 mL) were added DIAD (3.84 g, 18.987 mmol, 3 equiv.) dropwise at 0° C. under a nitrogen atmosphere. The resulting mixture was stirred for 2 h at room temperature under a nitrogen atmosphere. The resulting mixture was washed with brine (3×50 mL), dried over anhydrous Na₂SO₄ and filtered. The filtrate was concentrated under reduced pressure. The residue was purified by reversed-phase flash chromatography with the following conditions: column: C18 silica gel; mobile phase: MeCN in Water (0.1% NH3·H2O), 0% to 88% gradient in 10 min; detector: UV 254 nm to afford tert-butyl N-(4-{N-[(8S)-5,6,7,8-tetrahydroquinolin-8-yl]4-nitrobenzenesulfonamido}butyl)carbamate (3.1 g, 97.06%) as an off-white solid. MS (ESI): 505.0 [M+H]+.

### Step E6-3: Deprotection of Compound E52

To a stirred mixture of tert-butyl N-(4-{N-[(8S)-5,6,7,8-tetrahydroquinolin-8-yl]4-nitrobenzenesulfonamido}butyl)carbamate (3.1 g, 6.143 mmol, 1 equiv.) in DMF (30 mL) were added thioglycolic acid (2.83 g, 30.715 mmol, 5 equiv.) and DBU (4.68 g, 30.715 mmol, 5 equiv.) in portions at room temperature. The resulting mixture was stirred for 30 min at room temperature under an atmosphere of air. The reaction was quenched by the addition of water/ice (70 mL) at room temperature. The resulting mixture was extracted with EtOAc (3×80 mL). The combined organic layers were washed with brine (1×50 mL), dried over anhydrous Na₂SO₄, and filtered. After filtration, the filtrate was concentrated under reduced pressure. The residue was purified by reversed-phase flash chromatography with the following conditions: column: C18 silica gel; mobile phase: MeCN in Water (0.10% NH₃·H₂O), 0% to 86% gradient in 15 min; detector: UV 254 nm to afford tert-butyl N-{4-[(8S)-5,6,7,8-tetrahydroquinolin-8-ylamino]butyl}carbamate (1.6 g. 81.53%) as an off-white solid. MS (ESI): 320.0 [M+H]+.

### Step E6-4: Preparation of Compound E54.

A solution of tert-butyl N-{4-[(8S)-5,6,7,8-tetrahydroquinolin-8-ylamino]butyl}carbamate (1.6 g, 5.009 mmol, 1 equiv.) and 1-{[2-(trimethylsilyl)ethoxy]methyl}-1,3-benzodiazole-2-carbaldehyde (2.08 g, 7.514 mmol, 1.5 equiv.) in DCE (30 mL) was treated with AcOH (1.50 g, 25.045 mmol, 5 equiv.) for 30 min at room temperature under a nitrogen atmosphere followed by the addition of STAB (3.18 g, 15.027 mmol, 3 equiv.) in portions at room temperature. The resulting mixture was stirred for 2 h at room temperature under a nitrogen atmosphere. The resulting mixture was diluted with DCM (50 mL). The combined organic layers were washed with NaHCO₃ (3×50 mL), dried over anhydrous Na₂SO₄, and filtered. After filtration, the filtrate was concentrated under reduced pressure. The residue was purified by reversed-phase flash chromatography with the following conditions: column: C18 silica gel; mobile phase: MeCN in water (0.1% NH₃·H₂O), 0% to 100% gradient in 15 min; detector: UV 254 nm to afford tert-butyl N-{4-[(8S)-5,6,7,8-tetrahydroquinolin-8-yl[(1-{[2-(trimethylsilyl)ethoxy]methyl}-1,3-benzodiazol-2-yl)methyl]amino]butyl}carbamate (2.3 g, 79.19%) as a yellow green oil. MS (ESI): 579.0 [M+H]+.

### Step E6-5: Deprotection of Compound E54.

To a stirred solution of tert-butyl N-{4-[(8S)-5,6,7,8-tetrahydroquinolin-8-yl[(1-{[2-(trimethylsilyl)ethoxy]methyl}-1,3-benzodiazol-2-yl)methyl]amino]butyl}carbamate (2.3 g, 3.966 mmol, 1 equiv.) in DCM (10 mL) was added TFA (10 mL) dropwise at room temperature under an atmosphere of air. The resulting mixture was stirred overnight at room temperature under an atmosphere of air. The reaction was quenched by the addition of ice/NaHCO₃ salt (40 mL) at 0° C. The resulting mixture was extracted with EtOAc (5×30 mL). The combined organic layers were dried over anhydrous Na₂SO₄, and filtered. After filtration, the filtrate was concentrated under reduced pressure. The residue was purified by reversed-phase flash chromatography with the following conditions: column, C18 silica gel; mobile phase: MeCN in water (10 mmol/L NH₄HCO₃), 10% to 60% gradient in 20 min; detector: UV 254 nm to afford (8S)—N-(4-aminobutyl)-N-(1H-1,3-benzodiazol-2-ylmethyl)-5,6,7,8-tetrahydroquinolin-8-amine (1 g. 72.14%) as a white solid. MS (ESI): 350.1 [M+H]⁺. ¹H NMR (400 MHz, methanol-d4) δ 8.50 (dd, J=4.7, 1.7 Hz, 1), 7.55 (ddd, J=11.9, 5.9, 2.4 Hz, 3H), 7.27-7.15 (m, 3H), 4.13 (dd, J=9.8, 6.1 Hz, 1H), 4.10-3.96 (m, 2H), 2.87 (ddd, J=16.3, 10.8, 5.1 Hz, 1H), 2.76 (dt, J=17.0, 4.6 Hz, 1), 2.67-2.56 (m, 1), 2.56-2.38 (m, 3H), 2.29-2.19 (m, 1H), 2.09-1.90 (m, 2H), 1.70 (dtdd, J=13.3, 11.2, 5.0, 2.8 Hz, 1H), 1.45-1.29 (m, 4H).

We claim:

1. A method for preparing mavorixafor: 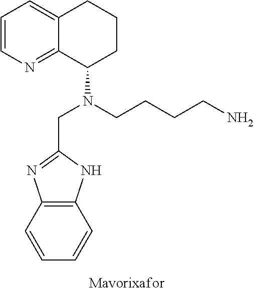 [SMILES: NCCCCN(Cc1nc2ccccc2[nH]1)[C@H]1CCCc2cccnc21]
or a pharmaceutically acceptable salt thereof;
comprising the steps of:
(a) asymmetrically coupling a compound of the formula A: 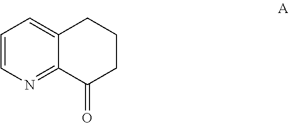 [SMILES: O=C1CCCc2cccnc21]
with a chiral compound to provide a compound of the formula D: 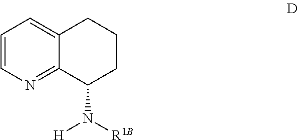
or a salt thereof, wherein:
R^{1B} is a chiral auxiliary;
(b) cleaving the chiral auxiliary R^{1B} to afford a compound of the formula E: 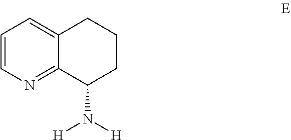 [SMILES: N[C@H]1CCCc2cccnc21]
or a salt thereof, wherein:
(c) coupling the compound of the formula E with a compound of the formula F: 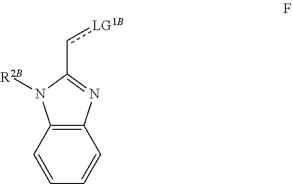
or a salt thereof, wherein:
R^{2B} is H, or a suitable benzimidazole protecting group; and
LG^{1B} is a suitable leaving group, ═O, or, taken with the carbon atom to which it is bound, an acetal;
to prepare a compound of the formula G: 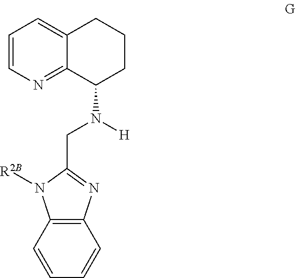
or a salt thereof;
(d) coupling the compound of formula G with a compound of the formula J: 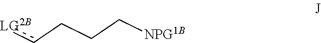
or a salt thereof, wherein:
LG^{2B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula K: 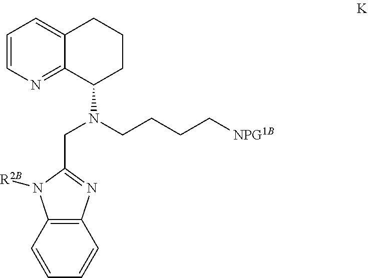
or a salt thereof;
(e) deprotecting the compound of formula K to form mavorixafor or a pharmaceutically acceptable salt thereof.

2. A method for preparing mavorixafor: 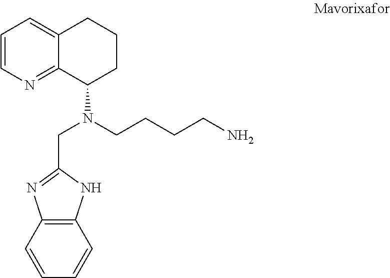 [SMILES: NCCCCN(Cc1nc2ccccc2[nH]1)[C@H]1CCCc2cccnc21]
or a pharmaceutically acceptable salt thereof;
comprising the steps of:
(a) asymmetrically coupling a compound of the formula A: 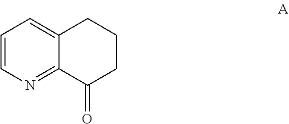 [SMILES: O=C1CCCc2cccnc21]
with a chiral compound to provide a compound of formula D: 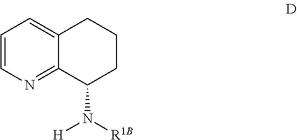
or a salt thereof, wherein:
R^{1B} is a chiral auxiliary;
(b) cleaving the chiral auxiliary R^{1B} to afford a compound of formula E: 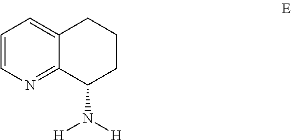 [SMILES: N[C@H]1CCCc2cccnc21]
or a salt thereof, wherein:
(c) coupling the compound of formula D with a compound of formula J: 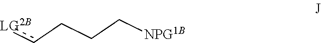
or a salt thereof, wherein:
LG^{2B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula G′: 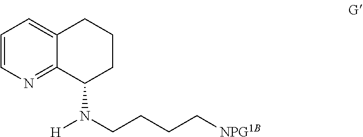
or a salt thereof;
(d) coupling of the compound of formula G′ with a compound of formula F: 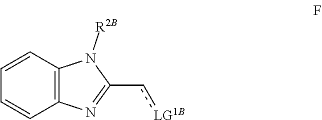
or a salt thereof, wherein:
R^{2B} is H, or a suitable benzimidazole protecting group; and
LG^{1B} is a suitable leaving group, ═O, or, taken with the carbon atom to which it is bound, an acetal;
to prepare a compound of the formula K: 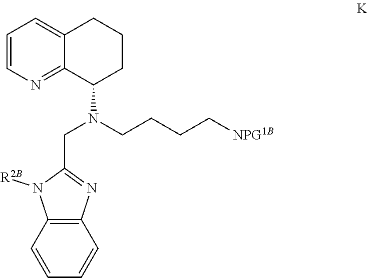
or a salt thereof;
(e) deprotecting the compound of formula K to form mavorixafor or a pharmaceutically acceptable salt thereof.

3. The method according to claim 1 or 2, wherein R^{1B}, taken with the nitrogen atom to which it is bound, is (S)-(−)-1-(4-methoxyphenyl)ethylamine: 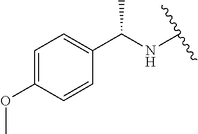 [SMILES: *N[C@@H](C)c1ccc(OC)cc1]
(S)-(−)-1-(4-methoxyphenyl)ethylamine.

4. The method according to claim 1 or 2, wherein R^{1B} is (S)-(−)-1-(4-methoxyphenyl)ethylamine, R^{2B} is SEM, LG^{1B} is ═O, LG^{2B} is ═O, and PG^{1B} is H and Boc.

5. The method according to claim 1, wherein the compound of formula E and the compound of formula F are coupled to prepare the compound of formula G through reductive amination.

6. The method according to claim 1, wherein the compound of formula G and the compound of formula J are coupled to prepare the compound of formula K through reductive amination.

7. The method according to claim 2, wherein the compound of formula E and the compound of formula J are coupled to prepare the compound of formula G′ through reductive amination.

8. The method according to claim 2, wherein the compound of formula F and the compound of formula G′ are coupled to prepare the compound of formula K through reductive amination.

9. A method for preparing mavorixafor: 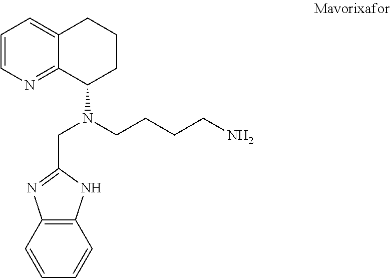 [SMILES: NCCCCN(Cc1nc2ccccc2[nH]1)[C@H]1CCCc2cccnc21]
or a pharmaceutically acceptable salt thereof;
comprising the steps of:
(a) coupling a compound of formula E: 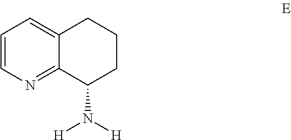 [SMILES: N[C@H]1CCCc2cccnc21]
or a salt thereof,
with a compound of formula F: 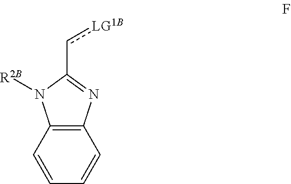
or a salt thereof, wherein:
R^{2B} is H, or a suitable benzimidazole protecting group; and
LG^{1B} is a suitable leaving group, ═O, or, taken with the carbon atom to which it is bound, an acetal;
to prepare a compound of formula G: 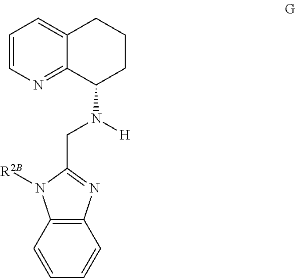
or a salt thereof;
(b) coupling the compound of formula G with a compound of formula J: 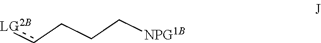
or a salt thereof, wherein:
LG^{2B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula K: 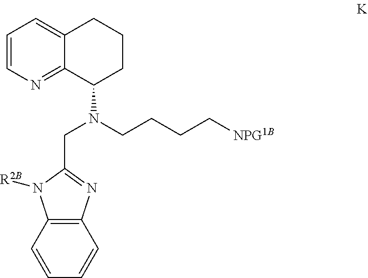
or a salt thereof; and
(c) deprotecting the compound of formula K to form mavorixafor or a pharmaceutically acceptable salt thereof.

10. A method for preparing mavorixafor: 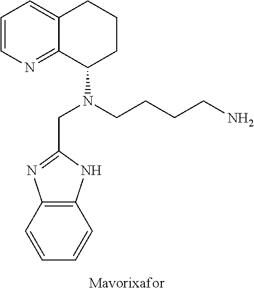 [SMILES: NCCCCN(Cc1nc2ccccc2[nH]1)[C@H]1CCCc2cccnc21]
or a pharmaceutically acceptable salt thereof;
comprising the steps of:
(a) coupling a compound of formula E: 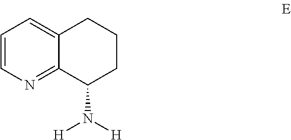 [SMILES: N[C@H]1CCCc2cccnc21]
or a salt thereof,
with a compound of formula J: 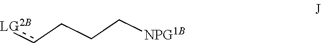
or a salt thereof, wherein:
LG^{2B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula G′: 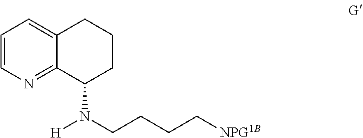
or a salt thereof;
(d) coupling the compound of formula G′ with a compound of formula F: 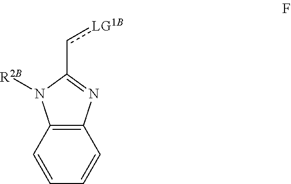
or a salt thereof;
wherein:
R^{2B} is H, or a suitable benzimidazole protecting group; and
LG^{1B} is a suitable leaving group, ═O, or, taken with the carbon atom to which it is bound, an acetal;
to prepare a compound of formula K: 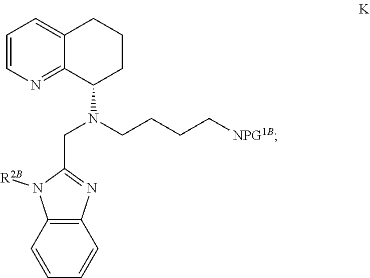
or a salt thereof; and
(e) deprotecting the compound of formula K to form mavorixafor or a pharmaceutically acceptable salt thereof.

11. The method according to claim 9 or 10, wherein LG^{1B} is ═O, LG^{2B} is ═O, R^{2B} is SEM, and PG^{1B} is Cbz and H.

12. The method according to claim 9, wherein the compound of formula E and the compound of formula F are coupled to prepare the compound of formula G through reductive amination.

13. The method according to claim 9, wherein the compound of formula G and the compound of formula J are coupled to prepare the compound of formula K through reductive amination.

14. The method according to claim 10, wherein the compound of formula E and the compound of formula J are coupled to prepare the compound of formula K through reductive amination.

15. The method according to claim 10, wherein the compound of formula G′ and the compound of formula F are coupled to prepare the compound of formula K through reductive amination.

16. A method for preparing mavorixafor: 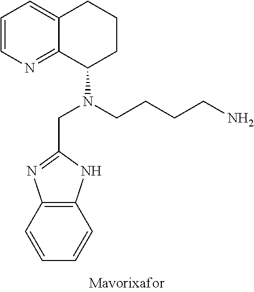 [SMILES: NCCCCN(Cc1nc2ccccc2[nH]1)[C@H]1CCCc2cccnc21]
or a pharmaceutically acceptable salt thereof, comprising the steps of:
(a) asymmetrically coupling a compound of formula A: 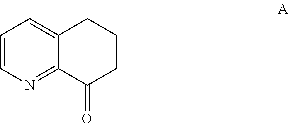 [SMILES: O=C1CCCc2cccnc21]
with a chiral amine to provide a compound of formula D: 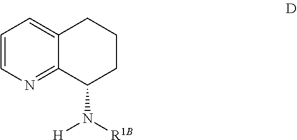
or a salt thereof, wherein:
R^{1B} is a chiral auxiliary;
(b) coupling the compound of formula D with a compound of formula F: 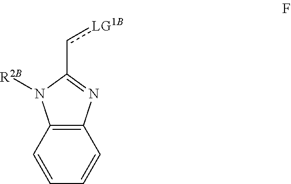
or a salt thereof, wherein:
R^{2B} is H, or a suitable benzimidazole protecting group; and
LG^{1B} is a suitable leaving group, ═O, or, taken with the carbon atom to which it is bound, an acetal;
to prepare a compound of formula G: 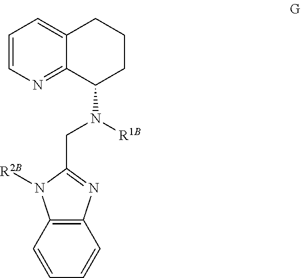
or a salt thereof;
(c) cleaving R^{1B} to prepare a compound of formula H: 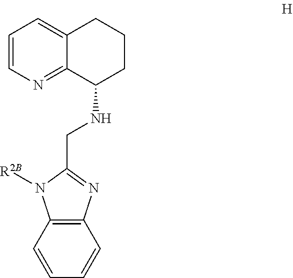
or a salt thereof;
(d) coupling the compound of formula H with a compound of formula J: 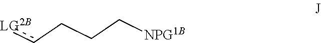
or a salt thereof,
wherein:
LG^{2B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula K: 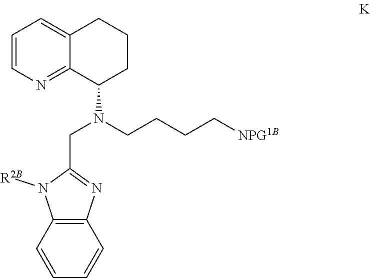
or a salt thereof;
(e) deprotecting the compound of formula K to form mavorixafor or a pharmaceutically acceptable salt thereof.

17. A method for preparing mavorixafor: 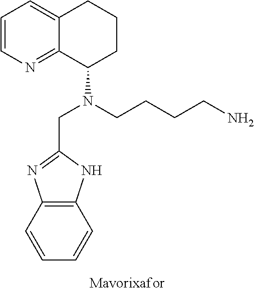 [SMILES: NCCCCN(Cc1nc2ccccc2[nH]1)[C@H]1CCCc2cccnc21]
or a pharmaceutically acceptable salt thereof, comprising the steps of:
(a) asymmetrically coupling a compound of formula A: 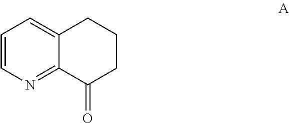 [SMILES: O=C1CCCc2cccnc21]
with a chiral amine to provide a compound of formula D: 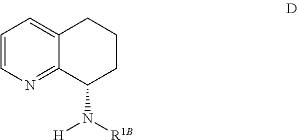
or a salt thereof,
wherein:
R^{1B} is a chiral auxiliary;
(f) coupling the compound of formula D with a compound of formula J: 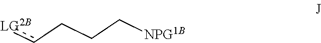
or a salt thereof,
wherein:
LG^{2B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula G′: 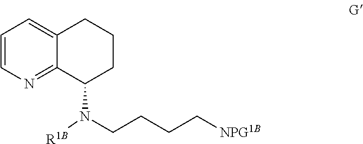
or a salt thereof;
(b) cleaving R^{1B} to prepare a compound of formula H′: 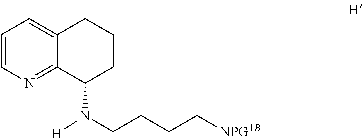
or a salt thereof,
(g) coupling the compound of formula H′ with the formula F: 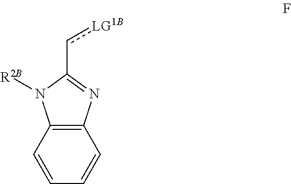
or a salt thereof,
wherein:
R^{2B} is H, or a suitable benzimidazole protecting group; and
LG^{1B} is a suitable leaving group, ═O, or, taken with the carbon atom to which it is bound, an acetal;
to prepare a compound of the formula K: 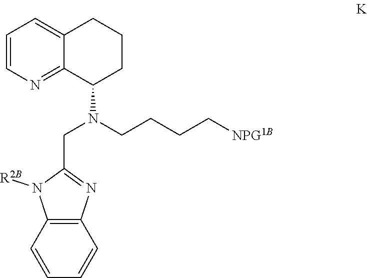
or a salt thereof; and
deprotecting the compound of formula K to form mavorixafor or a pharmaceutically acceptable salt thereof.

18. The method according to claim 16 or 17, wherein R^{1B}, taken with the nitrogen atom to which it is bound, is (S)-(−)-1-(4-methoxyphenyl)ethylamine:

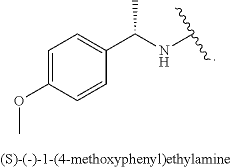 [SMILES: *N[C@@H](C)c1ccc(OC)cc1]

19. The method according to claim 16 or 17, wherein R^{1B}, taken with the nitrogen atom to which it is bound, (S)-(−)-1-(4-methoxyphenyl)ethylamine, LG^{1B} is chloro, R^{2B} is CH₂OCH₂CH₃, LG^{2B} is ═O, and PG^{1B} is Boc and Boc.

20. The method according to claim 16, wherein the compound of formula D and the compound of formula F are coupled to prepare the compound of formula G through reductive amination.

21. The method according to claim 16, wherein the compound of formula H and the compound of formula J are coupled to prepare the compound of formula K through reductive amination.

22. The method according to claim 17, wherein the compound of formula D and the compound of formula J are coupled to prepare the compound of formula G′ through reductive amination.

23. The method according to claim 17, wherein the compound of formula H′ and the compound of formula F are coupled to prepare the compound of formula K through reductive amination.

24. A method for preparing mavorixafor: 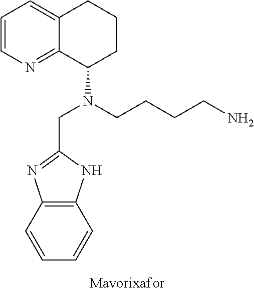 [SMILES: NCCCCN(Cc1nc2ccccc2[nH]1)[C@H]1CCCc2cccnc21]
or a pharmaceutically acceptable salt thereof;
comprising the steps of:
(a) condensing a compound of formula A: 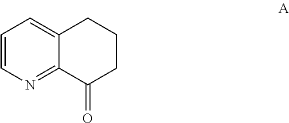 [SMILES: O=C1CCCc2cccnc21]
with a chiral compound to form a compound of formula L: 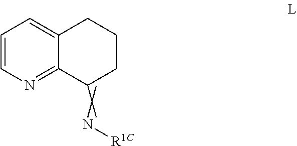
or a salt thereof,
wherein R^{1C} is a chiral auxiliary;
(b) asymmetrically reducing the compound of formula L to prepare a compound of formula M: 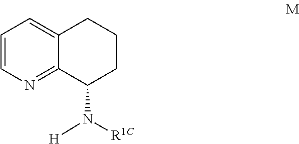
or a salt thereof;
(c) cleaving R^{1C} to prepare a compound of formula N: 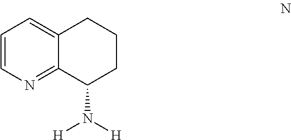 [SMILES: N[C@H]1CCCc2cccnc21]
or a salt thereof;
(d) protecting the compound of formula N to prepare a compound of formula O: 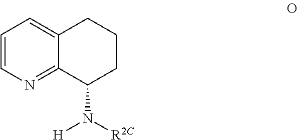
or a salt thereof,
wherein:
R^{2C} is H, or a suitable amino protecting group;
(e) coupling the compound of formula O to a compound of formula P: 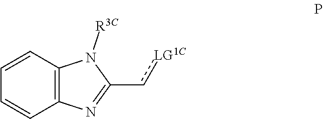
or a salt thereof,
wherein:
R^{3C} is H, or a suitable benzimidazole protecting group; and
LG^{1C} is a suitable leaving group, ═O, or, taken with the carbon atom to which it is bound, an acetal;
to prepare a compound of formula Q: 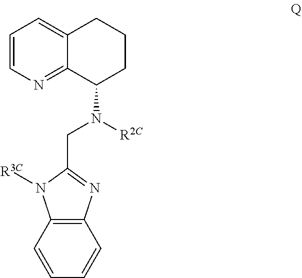
or salt thereof;
(f) deprotecting the compound of formula Q to prepare a compound of formula R: 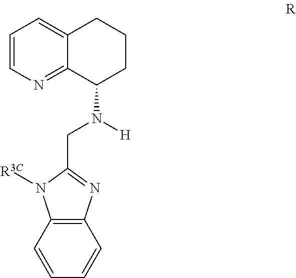
or a salt thereof;
(g) coupling the compound of formula R with a compound of formula S: 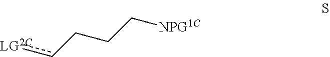
or a salt thereof,
wherein:
LG^{2C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of the formula T: 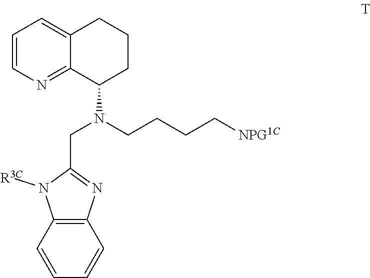
or a salt thereof; and
(h) deprotecting the compound of formula T to form mavorixafor or a pharmaceutically acceptable salt thereof.

25. A method for preparing mavorixafor: 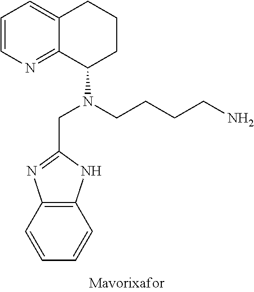 [SMILES: NCCCCN(Cc1nc2ccccc2[nH]1)[C@H]1CCCc2cccnc21]
or a pharmaceutically acceptable salt thereof;
comprising the steps of:
(a) condensing a compound of formula A: 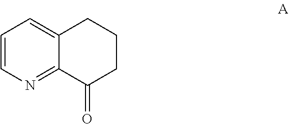 [SMILES: O=C1CCCc2cccnc21]
with a chiral compound to form a compound of formula L: 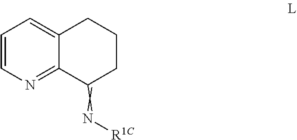
or a salt thereof, wherein R^{1C} is a chiral auxiliary;
(b) asymmetrically reducing the compound of formula L to prepare a compound of formula M: 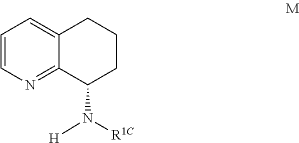
or a salt thereof;
(c) cleaving R^{1C} to prepare a compound of formula N: 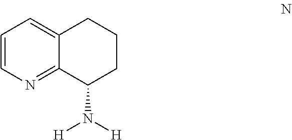 [SMILES: N[C@H]1CCCc2cccnc21]
or a salt thereof;
(d) protecting the compound of formula N to prepare a compound of formula O: 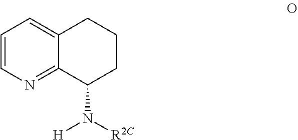
or a salt thereof,
wherein:
R^{2C} is H, or a suitable amino protecting group;
(e) coupling the compound of formula O with a compound of formula S: 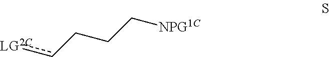
or a salt thereof,
wherein:
LG^{2C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of the formula Q′: 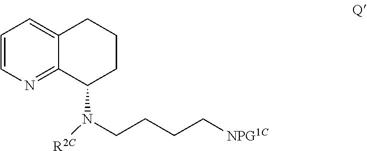
or a salt thereof;
(f) deprotecting the compound of formula Q′ to prepare a compound of the formula R′: 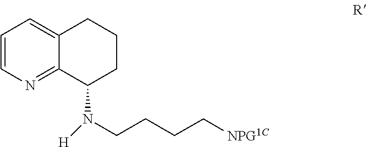
or a salt thereof;
(g) coupling the compound of formula R′ to a compound of formula P: 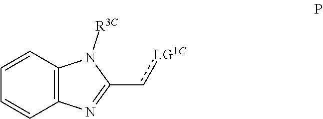
or a salt thereof, wherein:
R^{3C} is H, or a suitable benzimidazole protecting group; and
LG^{1C} is a suitable leaving group, ═O, or, taken with the carbon atom to which it is bound, an acetal;
to prepare a compound of formula T: 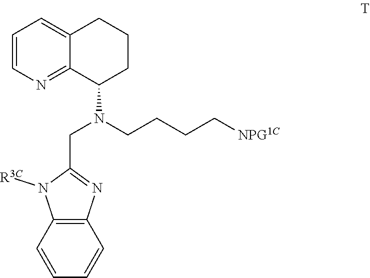
or salt thereof;
(h) deprotecting the compound of formula T to form mavorixafor or a pharmaceutically acceptable salt thereof.

26. A method for preparing mavorixafor: 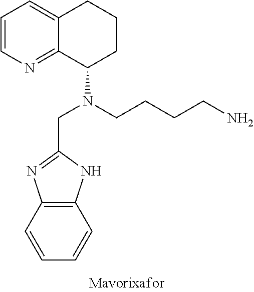 [SMILES: NCCCCN(Cc1nc2ccccc2[nH]1)[C@H]1CCCc2cccnc21]
or a pharmaceutically acceptable salt thereof;
comprising the steps of:
(a) condensing a compound of formula A: 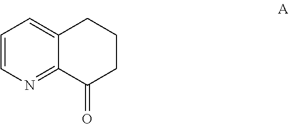 [SMILES: O=C1CCCc2cccnc21]
with a chiral compound to form a compound of formula L: 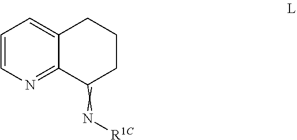
or a salt thereof,
wherein R^{1C} is a chiral auxiliary;
(b) asymmetrically reducing the compound of formula L to prepare a compound of formula M: 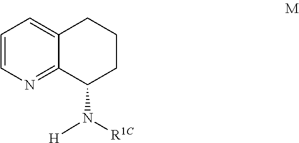
or a salt thereof;
(c) coupling the compound of formula M to a compound of formula P: 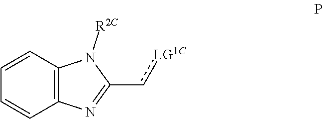
or a salt thereof,
wherein:
R^{2C} is H, or a suitable benzimidazole protecting group; and
LG^{1C} is a suitable leaving group, ═O, or, taken with the carbon atom to which it is bound, an acetal;
to prepare a compound of formula Q: 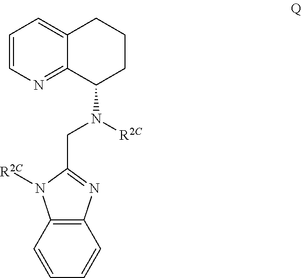
or salt thereof;
(d) deprotecting the compound of formula Q to prepare a compound of formula R: 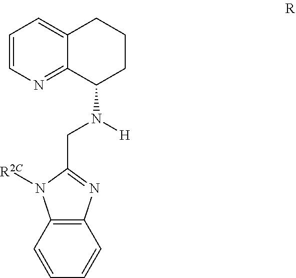
or a salt thereof;
(e) coupling the compound of formula R with a compound of formula S: 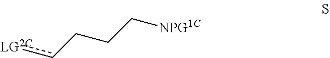
or a salt thereof,
wherein:
LG^{2C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of the formula T: 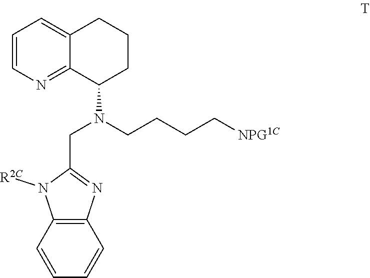
or a salt thereof; and
(f) deprotecting the compound of formula T to form mavorixafor or a pharmaceutically acceptable salt thereof.

27. A method for preparing mavorixafor: 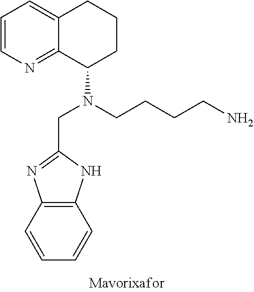 [SMILES: NCCCCN(Cc1nc2ccccc2[nH]1)[C@H]1CCCc2cccnc21]
or a pharmaceutically acceptable salt thereof;
comprising the steps of:
(a) condensing a compound of formula A: 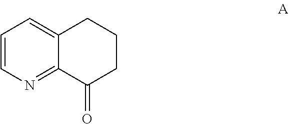 [SMILES: O=C1CCCc2cccnc21]
with a chiral compound to form a compound of formula L: 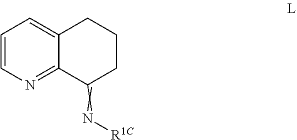
or a salt thereof, wherein R^{1C} is a chiral auxiliary;
(b) asymmetrically reducing the compound of formula L to prepare a compound of formula M: 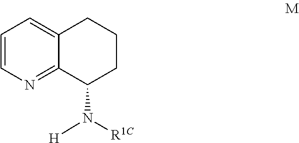
or a salt thereof;
(c) coupling the compound of formula M with a compound of formula S: 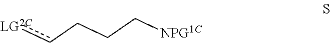
or a salt thereof,
wherein:
LG^{2C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of the formula Q′: 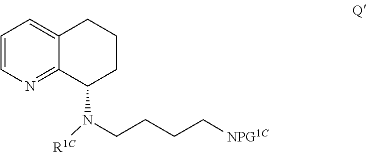
or a salt thereof;
(d) deprotecting the compound of formula Q′ to prepare a compound of the formula 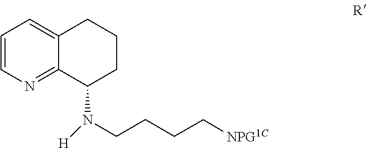
or a salt thereof;
(e) coupling the compound of formula R′ to a compound of formula P: 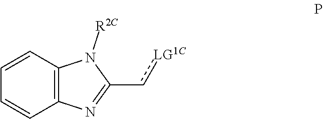
or a salt thereof, wherein:
R^{2C} is H, or a suitable benzimidazole protecting group; and
LG^{1C} is a suitable leaving group, ═O, or, taken with the carbon atom to which it is bound, an acetal;
to prepare a compound of formula T: 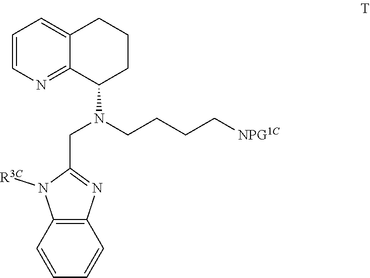
or salt thereof;
(f) deprotecting the compound of formula T to form mavorixafor or a pharmaceutically acceptable salt thereof.

28. The method according to claim 24, 25, 26 or 27, wherein R^{1C}, taken with the nitrogen atom to which it is bound, is (S)-(−)-2-methyl-2-propanesulfinamide:

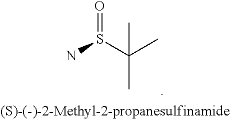

29. The method according to claim 24 or 25, wherein R^{1C}, taken with the nitrogen atom to which it is bound, is (S)-(−)-2-methyl-2-propanesulfinamide, R^{2C} is 4-nitrobenzenesulfonyl, LG^{1C} is hydroxyl, R^{3C} is SEM, LG^{2C} is ═O, and PG^{1C} is Boc and H.

30. The method according to claim 26 or 27, wherein R^{1C}, taken with the nitrogen atom to which it is bound, is (S)-(−)-2-methyl-2-propanesulfinamide, LG^{1C} is hydroxyl, R^{2C} is SEM, LG^{2C} is ═O, and PG^{1C} is Boc and H.

31. The method according to claim 24, wherein the compound of formula O and the compound of formula P are coupled to prepare the compound of formula Q through reductive amination.

32. The method according to claim 24, wherein the compound of formula R and the compound of formula S are coupled to prepare the compound of formula T through reductive amination.

33. The method according to claim 25, wherein the compound of formula O and the compound of formula S are coupled to prepare the compound of formula Q′ through reductive amination.

34. The method according to claim 25, wherein the compound of formula R′ and the compound of formula P are coupled to prepare the compound of formula T through reductive amination.

35. The method according to claim 26, wherein the compound of formula M and the compound of formula P are coupled to prepare the compound of formula Q through reductive amination.

36. The method according to claim 26, wherein the compound of formula R and the compound of formula S are coupled to prepare the compound of formula T through reductive amination.

37. The method according to claim 27, wherein the compound of formula M and the compound of formula S are coupled to prepare the compound of formula Q′ through reductive amination.

38. The method according to claim 27, wherein the compound of formula R′ and the compound of formula P are coupled to prepare the compound of formula T through reductive amination.

39. A method for preparing mavorixafor: 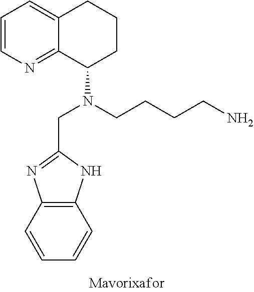 [SMILES: NCCCCN(Cc1nc2ccccc2[nH]1)[C@H]1CCCc2cccnc21]
or a pharmaceutically acceptable salt thereof;
comprising the steps of:
(a) protecting the compound with formula N: 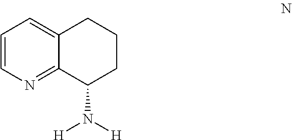 [SMILES: N[C@H]1CCCc2cccnc21]
or a salt thereof;
to prepare a compound of formula O: 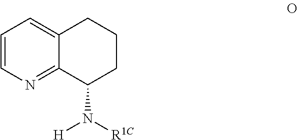
or a salt thereof;
wherein:
R^{1C} is H, or a suitable amino protecting group;
(b) coupling a compound of formula O to a compound of formula P: 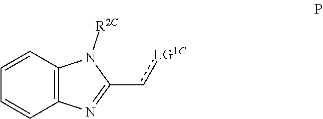
or a salt thereof;
wherein:
R^{2C} is H, or a suitable benzimidazole protecting group; and
LG^{1C} is a suitable leaving group, ═O, or, taken with the carbon atom to which it is bound, an acetal;
to prepare a compound of formula Q: 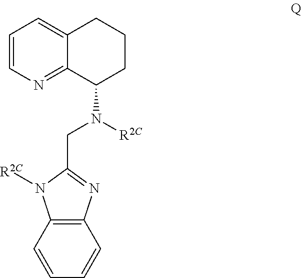
or salt thereof;
(c) deprotecting the compound of formula Q to prepare a compound of formula R: 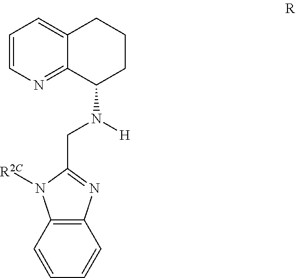
or a salt thereof;
(d) coupling the compound of formula R with a compound of formula S: 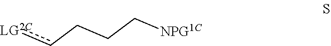
or a salt thereof;
wherein:
LG^{2C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of formula T: 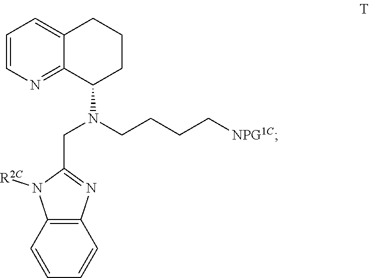
or a salt thereof; and
(e) deprotecting a compound of formula T to prepare mavorixafor or a salt thereof.

40. A method for preparing mavorixafor: 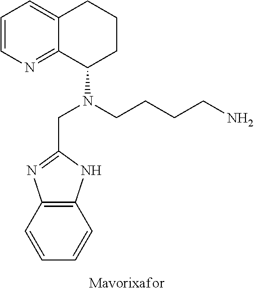 [SMILES: NCCCCN(Cc1nc2ccccc2[nH]1)[C@H]1CCCc2cccnc21]
or a pharmaceutically acceptable salt thereof;
comprising the steps of:
(a) protecting a compound of formula N: 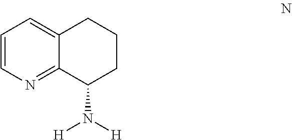 [SMILES: N[C@H]1CCCc2cccnc21]
or a salt thereof;
to prepare a compound of formula O: 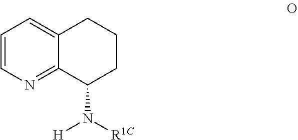
or a salt thereof;
wherein:
R^{1C} is H, or a suitable amino protecting group;
(b) coupling the compound of formula R with a compound of formula S: 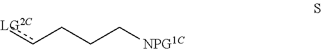
or a salt thereof;
wherein:
LG^{2C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of formula Q′: 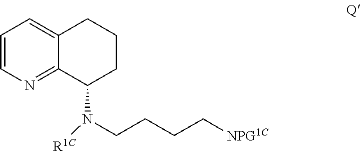
or a salt thereof;
(c) deprotecting the compound of formula Q to prepare a compound of formula R′: 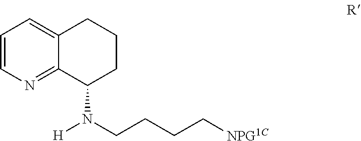
or a salt thereof;
(d) coupling the compound of formula R′ to a compound of formula P: 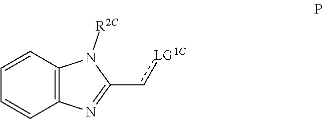
or a salt thereof:
wherein:
R^{2C} is H, or a suitable benzimidazole protecting group; and
LG^{1C} is a suitable leaving group, ═O, or, taken with the carbon atom to which it is bound, an acetal:
to prepare a compound of formula T: 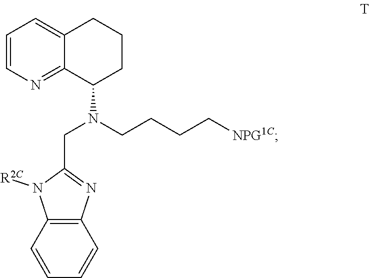
or a salt thereof; and
(e) deprotecting a compound of formula T to prepare mavorixafor or a salt thereof.

41. The method according to claim 39 or 40, wherein R^{1C} is 4-nitrobenzenesulfonyl, LG^{1C} is hydroxyl, R^{2C} is SEM, LG^{2C} is ═O, and PG^{1C} is Cbz and H.

42. The method according to claim 39 or 40, wherein R^{1C} is 4-nitrobenzenesulfonyl, LG^{1C} is ═O, R^{2C} is SEM, LG^{2C} is hydroxyl, and PG^{1C} is Boc and H.

43. The method according to claim 39, wherein the compound of formula O and the compound of formula P are coupled to prepare the compound of formula Q through reductive amination.

44. The method according to claim 39, wherein the compound of formula R and the compound of formula S are coupled to prepare the compound of formula T through reductive amination.

45. The method according to claim 40, wherein the compound of formula O and the compound of formula S are coupled to prepare the compound of formula Q′ through reductive amination.

46. The method according to claim 2, wherein the compound of formula R′ and the compound of formula P are coupled to prepare the compound of formula T through reductive amination.

47. A method for preparing mavorixafor: 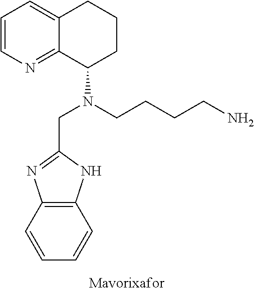 [SMILES: NCCCCN(Cc1nc2ccccc2[nH]1)[C@H]1CCCc2cccnc21]
or a pharmaceutically acceptable salt thereof;
comprising the steps of:
(a) protecting a compound of the formula U: 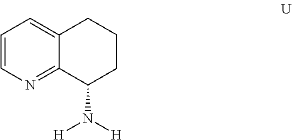 [SMILES: N[C@H]1CCCc2cccnc21]
or a salt thereof:
with a compound of the formula V: 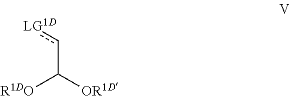
wherein R^{1D} and R^{1D′} are, taken together with the oxygen atoms to which they are bound and the carbon atom to which the oxygen atoms are bound, an acetal; and
LG^{1D} is a suitable leaving group, or ═O;
to prepare a compound of the formula W: 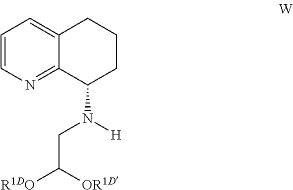
or a salt thereof;
(b) protecting the compound of formula W to prepare a compound of the formula X: 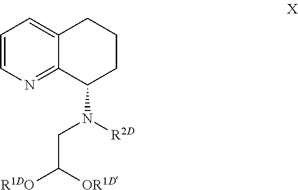
or a salt thereof;
wherein:
R^{2D} is H, or a suitable amino protecting group;
(c) coupling the compound of the formula X with a compound of the formula Y: 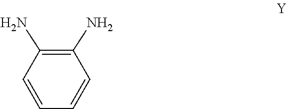
or a salt thereof:
to prepare a compound of the formula Z: 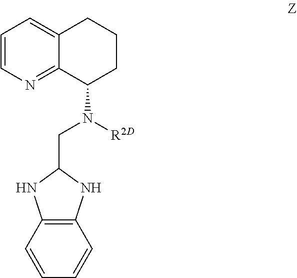
or a salt thereof;
(d) oxidizing the compound of formula Z to prepare a compound of the formula AA: 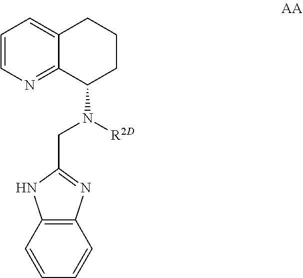
or a salt thereof;
(e) protecting the compound of formula AA to prepare a compound of the formula AB: 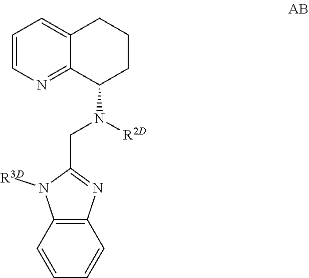
or a salt thereof;
wherein:
R^{3D} is H, or a suitable benzimidazole protecting group;
(f) deprotecting the compound of formula AB to prepare a compound of the formula AC: 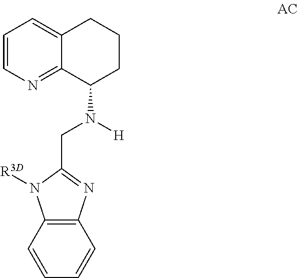
or a salt thereof:
(g) coupling the compound of formula AC with a compound of the formula AD: 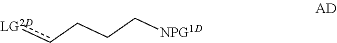
or a salt thereof;
wherein:
LG^{2D} is a suitable leaving group, ═O, or, taken with the carbon atom to which it is bound, an acetal; and
PG^{1D} is any combination of two amino protecting groups, any single amino protecting group and H, or two H; or PG^{1D} is, taken with the nitrogen to which it is bound, a cyclic imide; or PG^{1D} is absent and a triple bond is present between the corresponding nitrogen and the carbon to which the nitrogen is bound such that a nitrile is present;
to prepare a compound of the formula AE: 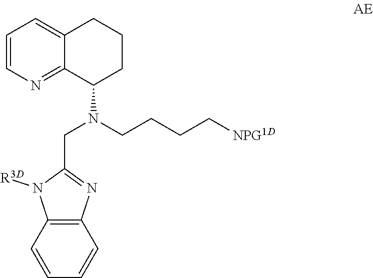
or a salt thereof; and
(h) deprotecting the compound of formula AE to prepare mavorixafor, or a salt thereof.

48. A method for preparing mavorixafor: 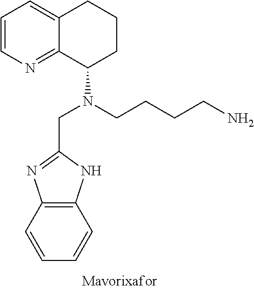 [SMILES: NCCCCN(Cc1nc2ccccc2[nH]1)[C@H]1CCCc2cccnc21]
or a pharmaceutically acceptable salt thereof;
comprising the steps of:
(a) protecting a compound of the formula U: 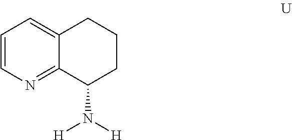 [SMILES: N[C@H]1CCCc2cccnc21]
or a salt thereof:
to prepare a compound of the formula W′: 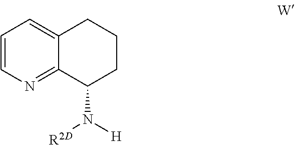
or a salt thereof;
wherein: R^{2D} is H, or a suitable amino protecting group;
(b) coupling the compound of formula W′ with a compound of the formula AD: 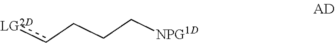
or a salt thereof;
wherein:
LG^{2D} is a suitable leaving group, ═O, or, taken with the carbon atom to which it is bound, an acetal; and
PG^{1D} is any combination of two amino protecting groups, any single amino protecting group and H, or two H; or PG^{1D} is, taken with the nitrogen to which it is bound, a cyclic imide; or PG^{1D} is absent and a triple bond is present between the corresponding nitrogen and the carbon to which the nitrogen is bound such that a nitrile is present;
to prepare a compound of the formula X′: 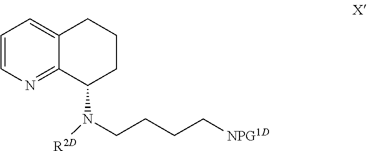
or salt thereof;
(c) deprotecting the compound of formula X′ to prepare a compound of the formula Z′; 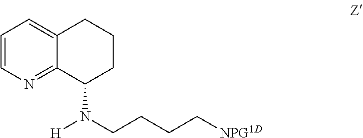
or salt thereof;
(d) coupling the compound of formula Z′ with a compound of the formula V: 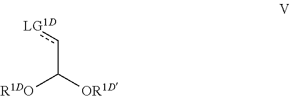
wherein R^{1D} and R^{1D′} are, taken together with the oxygen atoms to which they are bound and the carbon atom to which the oxygen atoms are bound, an acetal; and
LG^{1D} is a suitable leaving group, or =0;
to prepare a compound of the formula AA′: 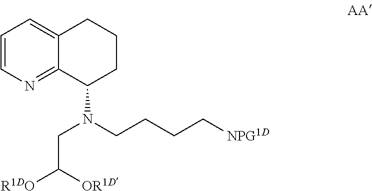
or a salt thereof;
(e) coupling the compound of formula AA′ with a compound of the formula Y: 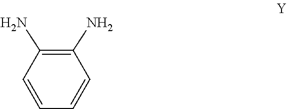
or a salt thereof;
to prepare a compound of the formula AB′: 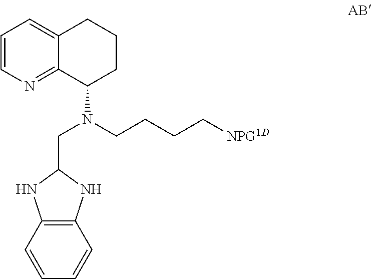
or salt thereof;
(f) oxidizing the compound of formula AB′ to prepare a compound of the formula AC′ 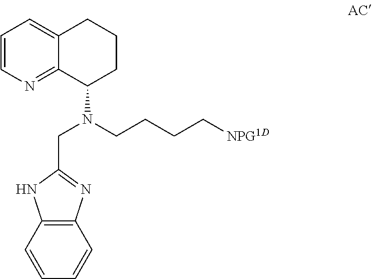
or salt thereof;
(g) deprotecting the compound of formula AC′ to prepare mavorixafor or a salt thereof.

49. The method according to claim 47, wherein the compound of formula U and the compound of formula V are coupled to prepare the compound of formula W through reductive amination.

50. The method according to claim 47, wherein the compound of formula AC and the compound of formula AD are coupled to prepare the compound of formula AE through reductive amination.

51. The method according to claim 48, wherein the compound of formula W′ and the compound of formula AD are coupled to prepare the compound of formula X′ through reductive amination.

52. The method according to claim 48, wherein the compound of formula Z′ and the compound of formula V are coupled to prepare the compound of formula AA′ through reductive amination.

53. The method according to claim 47, wherein RD and R^{1D′} are each ethyl; LG^{1D} is bromo; R^{2D} is Cbz; R^{3D} is SEM; LG^{2D} is ═O; and PG^{1D} is H and Boc.

54. The method according to claim 47, wherein R^{1D} and R^{1D′} are, taken with the oxygen to which they are bound and the carbon to which the oxygen atoms are bound, 1,3-dioxolane; LG^{1D} is bromo: R^{2D} is Cbz: R^{3D} is SEM: LG^{2D} is ═O; and PG^{1D} is H and Boc.

55. The method according to claim 48, wherein R^{1D} and R^{1D′} are each ethyl; LG^{1D} is bromo; R^{2D} is Cbz; LG^{2D} is ═O; and PG^{1D} is H and Boc.

56. The method according to claim 48, wherein R^{1D} and R^{1D′} are, taken with the oxygen to which they are bound and the carbon to which the oxygen atoms are bound, 1,3-dioxolane; LG^{1D} is bromo; R^{2D} is Cbz; LG^{2D} is ═O; and PG^{1D} is H and Boc.
